(19) United States
(12) Patent Application Publication
(10) Pub. No.: US 2017/0356032 A1
(43) Pub. Date: Dec. 14, 2017

(54) DIFFERENTIAL DIAGNOSIS AND THERAPY SELECTION FOR RHEUMATOID ARTHRITIS AND PSORIATIC ARTHRITIS

(71) Applicant: Fundacio Hospital Universitari Vall D'Hebron-Institut De Recerca, Barcelona (ES)

(72) Inventors: Sara Marsal BARRIL, Barcelona (ES); Antonio Julia CANO, Barcelona (ES)

(21) Appl. No.: 15/417,649

(22) Filed: Jan. 27, 2017

Related U.S. Application Data
(63) Continuation of application No. PCT/IB2015/055739, filed on Jul. 29, 2015.
(60) Provisional application No. 62/030,511, filed on Jul. 29, 2014.

(51) Int. Cl. C12Q 1/68; A61K 31/33

(52) U.S. Cl. CPC C12Q 1/6827; A61K 31/33; C12Q 1/6816; G01N 2800/102

(57) ABSTRACT

The present invention provides methods for differentially diagnosing rheumatoid arthritis (RA) versus psoriatic arthritis (PsA) in an individual. Specifically, the method relates to detecting the presence or absence of distinct alleles of the PDE4D and PDE4B genes which are associated with either RA or PsA. Also provided herein are methods for selecting an individual who should receive or who is likely to respond to a treatment with a PDE4 inhibitor. The present invention also provides methods for treating an individual with RA or PsA.

### CROSS-REFERENCES TO RELATED APPLICATIONS

This application is a continuation of PCT Application No. PCT/IB2015/055739, filed Jul. 29, 2015, which claims priority to U.S. Provisional Application No. 62/030,511, filed Jul. 29, 2014, the disclosures of which are hereby incorporated by reference in their entirety for all purposes.

### BACKGROUND OF THE INVENTION

Rheumatoid arthritis (RA) and psoriatic arthritis (PsA) are both autoimmune diseases in which the body's own immune system attacks itself. While both diseases involve joint inflammation, the other symptoms of the diseases and their courses of disease progression are significant different. For example, rheumatoid arthritis is more commonly seen in younger patients, often developing in the early 20s, and more often in women. Psoriatic arthritis, however, strikes members of both sexes equally and tends to have a later onset. In addition, rheumatoid arthritis is generally a symmetric disease, affecting both sides of the body equally. Hands and wrists are typically the most affected by rheumatoid arthritis. Psoriatic arthritis, on the other hand, is asymmetric in its distribution. While it also affects hands, it tends to affect only one side and is also often seen in the feet.

Nevertheless, rheumatoid arthritis and psoriatic arthritis can look very similar on the surface, making it difficult for physicians to make an accurate diagnosis. Indeed, making a correct diagnosis of rheumatoid arthritis and psoriatic arthritis is complex, particularly at the early stages of the disease. Commonly used biomarkers such as rheumatoid factor (RF) and anti-cyclic citrullinated protein antibodies (ACPAs) are not sufficient to diagnose these diseases or differentiate between them. Moreover, due to the fundamental differences between these two diseases, it is important to make a correct diagnosis to determine the correct treatment. As such, there is a need in the art for novel genetic markers that can aid or assist in improving the diagnostic process at the early stages of rheumatoid arthritis and psoriatic arthritis when both types of arthritis are less differentiated and more difficult to diagnose. Similarly, there is a need in the art for genetic markers that aid or assist in discriminating the likelihood of response to treatment for rheumatoid arthritis or psoriatic arthritis, particularly for drugs that may vary in their effectiveness for treating these diseases. The present invention satisfies these needs and provides related advantages as well.

### BRIEF SUMMARY OF THE INVENTION

In one aspect, the present invention provides a method for aiding or assisting in the differential diagnosis of rheumatoid arthritis (RA) versus psoriatic arthritis (PsA) in an individual. The method comprises:
(a) detecting the presence of a single nucleotide polymorphism (SNP) in a cAMP-specific 3′,5′-cyclic phosphodiesterase 4 (PDE4) gene selected from the group consisting of a PDE4D gene, PDE4B gene, and combinations thereof in a sample obtained from the individual, wherein the SNP in the PDE4D gene comprises rs7733296 and the SNP in the PDE4B gene comprises rs9326069; and
(b) aiding or assisting in the diagnosis of (i) RA based on the presence of an A allele or a complementary allele thereof (i.e., a T allele) at rs7733296 and/or a G allele or a complementary thereof (i.e., a C allele) at rs9326069, or (ii) PsA based on the presence of a G allele or a complementary allele thereof (i.e., a C allele) at rs7733296 and/or an A allele or a complementary allele thereof (i.e., a T allele) at rs9326069.

In some embodiments, the diagnosis of RA is based on the presence of two A alleles or complementary alleles thereof at rs7733296 and/or two G alleles or complementary alleles thereof at rs9326069. In some instances, step (a) of the method comprises detecting the presence of a SNP allele in the PDE4D gene and a SNP allele in the PDE4B gene. The method can include detecting the presence of one or two SNP alleles in the PDE4D gene (i.e., rs7733296) and one or two SNP alleles in the PDE4B gene (i.e., rs9326069).

In some embodiments, the method improves the diagnosis of early stage RA or early stage PsA. In certain embodiments, the method aids or assists in the diagnosis of RA by indicating, predicting, or determining an increased likelihood of having RA or an increased susceptibility to developing RA based on the presence of an A allele or a complementary allele thereof at rs7733296 and/or a G allele or a complementary allele thereof at rs9326069. In certain other embodiments, the method aids or assists in the diagnosis of PsA by indicating, predicting, or determining an increased likelihood of having PsA or an increased susceptibility to developing PsA based on the presence of a G allele or a complementary allele thereof at rs7733296 and/or an A allele or a complementary allele thereof at rs9326069.

In some embodiments, the method further comprises detecting the presence of an allele (i.e., allele 1 or allele 2) of one or more SNPs in Table 1, e.g., 1, 2, 3, 4, 5, 6, 7, 8, 9, 10 or more SNPs in Table 1. In other embodiments, detecting the presence of an allele of one or more SNPs in Table 1 is performed as an alternative to detecting the presence of a SNP in the PDE4D gene, PDE4B gene or both. In some embodiments, the method further comprises detecting the presence or level of a serological marker in a sample obtained from the individual, detecting the presence or status of a clinical factor for the individual, or combinations thereof. In some instances, the serological marker is selected from the group consisting of rheumatoid factor (RF), an anti-citrullinated protein antibody, C-reactive protein (CRP) and combinations thereof. The presence or level of one or more serological markers, e.g., 1, 2, 3 or more serological markers can be determined. In some instances, the clinical factor is selected from the group consisting of the age of the individual, the sex of the individual, smoking history of the individual, a diagnosis of psoriasis, erythrocyte sedimentation rate (ESR), and combinations thereof. In some embodiments, the presence or status of one or more clinical markers, e.g., 1, 2, 3, 4, 5 or more clinical markers is determined.

In some embodiments, the method further comprises applying a statistical analysis to the presence of the SNP(s) in the PDE4 gene in combination with the presence of an allele of one or more SNPs in Table 1, the presence or level of one or more serological markers and/or the presence or status of one or more clinical factors. The statistical analysis can improve the sensitivity, specificity, and/or overall accuracy of the differential diagnosis of RA versus PsA. In some instances, the statistical analysis can improve the sensitivity, specificity, and/or overall accuracy of diagnosing early stage RA over standard diagnostic methods such as the ACR/EULAR classification criteria or diagnosing early stage PsA over standard diagnostic methods such as the CASPAR classification criteria.

In some embodiments, the sample is selected from the group consisting of whole blood, plasma, serum, synovial fluid, saliva, and urine. In preferred instances, the sample is whole blood, plasma or serum.

In a second aspect, the present invention provides a method for selecting an individual with rheumatoid arthritis (RA) or psoriatic arthritis (PsA) for treatment with a cAMP-specific 3′,5′-cyclic phosphodiesterase 4 (PDE4) inhibitor. The method comprises:
(a) detecting the presence of a single nucleotide polymorphism (SNP) in a PDE4 gene selected from the group consisting of a PDE4D gene, PDE4B gene, and combinations thereof in a sample obtained from the individual, wherein the SNP in the PDE4D gene comprises rs7733296 and the SNP in the PDE4B gene comprises rs9326069; and
(b) selecting the individual for treatment with the PDE4 inhibitor based on the presence of a G allele or a complementary allele thereof at rs7733296 and/or an A allele or a complementary allele thereof at rs9326069.

In some instances, step (a) of the method comprises detecting the presence of a SNP allele in the PDE4D gene (i.e., rs7733296) and a SNP allele in the PDE4B gene (i.e., rs9326069).

In some embodiments, the individual with RA or PsA is selected for treatment with a PDE4 inhibitor based on the presence of two G alleles or the complementary alleles thereof at rs7733296 and/or two A alleles or the complementary alleles thereof at rs9326069.

In some embodiments, the PDE4 inhibitor is selected from the group consisting of apremilast, cilomilast, diazepam, ibudilast, luteolin, mesembrenone, piclamilast, roflumilast, rolipram, and combinations thereof. In particular embodiments, the PDE4 inhibitor is apremilast.

In some embodiments, the method further comprises detecting the presence of an allele of one or more SNPs in Table 1, e.g., 1, 2, 3, 4, 5, 6, 7, 8, 9, 10 or more SNPs in Table 1. Alternatively, the presence of an allele of one or more SNPs in Table 1 is detected instead of a SNP in the PDE4D gene, PDE4B gene or both.

In some embodiments, the method further comprises detecting the presence or level of a serological marker in a sample obtained from the individual, detecting the presence or status of a clinical factor for the individual, or combinations thereof. In some instances, the serological marker is selected from the group consisting of rheumatoid factor (RF), an anti-citrullinated protein antibody, C-reactive protein (CRP) and combinations thereof. In some embodiments, the presence or level of one or more serological markers, e.g., 1, 2, 3 or more serological markers is determined. In some instances, the clinical factor is selected from the group consisting of the age of the individual, the sex of the individual, smoking history of the individual, a diagnosis of psoriasis, erythrocyte sedimentation rate (ESR), and combinations thereof. In some embodiments, the presence or status of one or more clinical markers, e.g., 1, 2, 3, 4, 5 or more clinical markers is determined.

In some embodiments, the method further comprises applying a statistical analysis to the presence of the specific SNP(s) in the PDE4 gene in combination with the presence of a specific allele of one or more SNPs in Table 1, the presence or level of one or more serological markers and/or the status, presence or level of one or more clinical factors. The statistical analysis may improve the sensitivity, specificity, and/or overall accuracy of therapy selection with the PDE4 inhibitor compared to other methods known to those of ordinary skill in the art.

In some embodiments, the sample is selected from the group consisting of whole blood, plasma, serum, synovial fluid, saliva, and urine. In particular instances, the sample is whole blood, plasma or serum.

In a third aspect, the present invention provides a method for determining the likelihood that an individual with rheumatoid arthritis (RA) or psoriatic arthritis (PsA) will respond to treatment with a cAMP-specific 3′,5′-cyclic phosphodiesterase 4 (PDE4) inhibitor. The method comprises:
(a) detecting the presence of a single nucleotide polymorphism (SNP) in a PDE4 gene selected from the group consisting of a PDE4D gene, PDE4B gene, and combinations thereof in a sample obtained from the individual, wherein the SNP in the PDE4D gene comprises rs7733296 and the SNP in the PDE4B gene comprises rs9326069; and
(b) determining that the individual has a higher likelihood of response to treatment with the PDE4 inhibitor based on the presence of a G allele or a complementary allele thereof at rs7733296 and/or an A allele or a complementary allele thereof at rs9326069.

In some instances, step (a) of the method comprises detecting the presence of a SNP allele in the PDE4D gene (i.e., rs7733296) and a SNP allele in the PDE4B gene (i.e., rs9326069).

In some embodiments, the individual with RA or PsA has a higher likelihood of response to treatment with a PDE4 inhibitor based on the presence of two G alleles or the complementary alleles thereof at rs7733296 and/or two A alleles or the complementary alleles thereof at rs9326069.

In some embodiments, the PDE4 inhibitor is selected from the group consisting of apremilast, cilomilast, diazepam, ibudilast, luteolin, mesembrenone, piclamilast, roflumilast, rolipram, and combinations thereof. In particular embodiments, the PDE4 inhibitor is apremilast.

In some embodiments, the method further comprises detecting the presence of an allele of one or more SNPs in Table 1, e.g., 1, 2, 3, 4, 5, 6, 7, 8, 9, 10 or more SNPs in Table 1. Optionally, rather than detecting the presence of a SNP at rs7733296 and/or a SNP at rs9326069, the method for determining likelihood of response to a PDE4 inhibitor includes detecting the presence of an allele of one or more SNPs in Table 1, e.g., 1, 2, 3, 4, 5, 6, 7, 8, 9, 10 or more SNPs in Table 1.

In other embodiments, the method further comprises detecting the presence or level of a serological marker in a sample obtained from the individual, detecting the s presence or status of a clinical factor for the individual, or combinations thereof. In some instances, the serological marker is selected from the group consisting of rheumatoid factor (RF), an anti-citrullinated protein antibody, a C-reactive protein (CRP) and combinations thereof. The presence or level of one or more serological markers, e.g., 1, 2, 3 or more serological markers can be determined. In some instances, the clinical factor is selected from the group consisting of the age of the individual, the sex of the individual, smoking history of the individual, a diagnosis of psoriasis, erythrocyte sedimentation rate (ESR), and combinations thereof. The presence or status of one or more clinical factors, e.g., 1, 2, 3, 4, 5 or more clinical factors can be determined.

In some embodiments, the method further includes applying a statistical analysis to the presence or absence of the SNP(s) in the PDE4 gene in combination with the presence of an allele of one or more SNPs in Table 1, the presence or level of one or more serological markers and/or the presence or status of one or more clinical factors. The statistical analysis can improve the sensitivity, specificity, and/or overall accuracy of discriminating the likelihood of response to treatment with the PDE4 inhibitor.

In some embodiments, the sample is selected from the group consisting of whole blood, plasma, serum, synovial fluid, saliva, and urine. In particular instances, the sample is whole blood, plasma or serum.

In a fourth aspect, the present invention provides a method for treating a human subject having RA that includes administering a therapeutically effective amount of a PDE4 inhibitor to the human subject having a G allele or a complementary allele thereof at rs7733296 and/or an A allele or a complementary allele thereof at rs9326069 and suffering from RA. The subject can be heterozygous or homozygous for the G allele (i.e., GG or GA) or the complementary allele thereof at rs7733296. In some cases, the subject is heterozygous or homozygous for the A allele (i.e., AA or AG) or the complementary allele thereof at the rs9326069. Alternatively, the subject is heterozygous or homozygous for the G allele or the complementary allele thereof at rs7733296 and heterozygous or homozygous for the A allele or the complementary allele thereof at rs9326069. In some embodiments, the PDE4 inhibitor is selected from the group consisting of apremilast, cilomilast, diazepam, ibudilast, luteolin, mesembrenone, piclamilast, roflumilast, rolipram, and combinations thereof. The PDE4 inhibitor can be apremilast. In some instances, the subject has early stage RA.

In some embodiments, if the human subject is homozygous for the A allele (i.e., AA) or the complementary allele thereof (i.e., TT) at rs7733296 and/or homozygous for the G allele (i.e., GG) or the complementary allele thereof (i.e., CC) at rs9326069, the subject is administered a therapeutically effective amount of an anti-TNFα inhibitor therapy such as, but not limited to, etanercept (ENBREL™), adalimumab (HUMIRA™), infliximab (REMICADE™), golimumab (SIMPONI®), certolizumag pegol)(CIMZIA®), ESBA105, and pegsunercept. In other embodiments, the subject is administered a therapeutically effective amount of a therapy selected from the group consisting of ustekinumab)(STELARA®), sirukumab, sarilumab, secukinumab, ocrelizumab, rituximab)(RITUXIN®), tocilizumab)(ACTEIVIRA®), ofatumumab (ARZERRA®), denosumab)(XGEVA®), abatacept (ORENCIA®), masitinib, baricitinib, tofacitinib (XEJLANZ®), anakinra (KINERET®), ABP 501 (Amgen), azathioprine (IMURAN®), cyclosporine, methotrexate, lefunomide, sulfasalazine, retinoid, corticosteroid, Cox-2 inhibitor, another nonsteroidal anti-inflammatory drug, and any combination thereof.

In a fifth aspect, the present invention provides a method for treating a human subject having PsA comprising administering a therapeutically effective amount of a PDE4 inhibitor to a human subject having a G allele or a complementary allele thereof at rs7733296 and/or an A allele or a complementary allele thereof at rs9326069 and suffering from PsA. The subject can be heterozygous or homozygous for the G allele (i.e., GA or GG) or a complementary allele thereof at rs7733296. In some cases, the subject is heterozygous or homozygous for the A allele (i.e., AG or AA) or a complementary allele thereof at rs9326069. Alternatively, the subject is heterozygous or homozygous for the G allele or a complementary allele thereof at rs7733296 and heterozygous or homozygous for the A allele or a complementary allele thereof at rs9326069. In some embodiments, the PDE4 inhibitor is selected from the group consisting of apremilast, cilomilast, diazepam, ibudilast, luteolin, mesembrenone, piclamilast, roflumilast, rolipram, and combinations thereof. The PDE4 inhibitor can be apremilast. In some instances, the subject has early stage PsA.

Other objects, features, and advantages of the present invention will be apparent to one of skill in the art from the following detailed description.

### DETAILED DESCRIPTION OF THE INVENTION

### I. Introduction

The present invention is based, in part, on the discovery of allelic variants at the PDE4B and PDE4D genes that are differentially associated with rheumatoid arthritis versus psoriatic arthritis. In some aspects, the presence of one or two G alleles or complementary alleles thereof of the SNP corresponding to rs7733296 and/or one or two A alleles or complementary alleles thereof of the SNP corresponding to rs9326069 indicates or predicts an increased likelihood of having PsA or susceptibility to PsA. In other aspects, the presence of two A alleles or complementary alleles thereof at rs7733296 and/or two G alleles or complementary alleles thereof at rs9326069 indicates or predicts an increased likelihood of having RA or susceptibility of RA. The methods provided herein are useful for diagnosing RA including early stage RA or PsA including early stage PsA. In certain aspects, a patient having a G allele or the complementary allele thereof at rs7733296 and/or an A allele at rs9326069 is also selected to receive a treatment comprising a PDE4 inhibitor. In some cases, the patient is heterozygous or homozygous for the G allele at rs7733296 and/or heterozygous or homozygous for the A allele at rs9326069. Such a patient is predicted to have a higher likelihood of response to PDE4 inhibitor therapy. The methods disclosed herein are useful for treating a patient with RA or a patient with PsA.

rs7733296 corresponds to an A/G SNP (or the complement thereof such as a T/C SNP) located within an intron of the human PDE4D gene (Gene ID No. 5144). The rs7733296 polymorphic site is located on chromosome 5 at chromosome position 59542312 of GRCh38.p2, which is position 6432505 of contig NT_034772.7.

rs9326069 corresponds to an A/G SNP (or the complement thereof such as a T/C SNP) located within an intron of the human PDE4B gene (Gene ID No. 5142). The rs9326069 polymorphic site is located on chromosome 1 at chromosomal position 65791733 of GRCh38, which is position 65205745 of contig NT_032977.10.

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “rheumatoid arthritis” or “RA” includes an autoimmune disease that causes chronic inflammation of the connective tissues in the body, most particularly, the joints and the tissue around the joints. In rheumatoid arthritis, multiple joints are usually inflamed in a symmetrical pattern (e.g., both sides of the body are affected). The term “rheumatoid arthritis” also includes conditions such as Sjögren's syndrome, where the inflammation affects organs and areas of the body other than the joints, e.g., the glands of the eyes and mouth, causing dryness of these areas.

The term “early stage RA” refers to an initial stage of rheumatoid arthritis characterized by the onset of symptoms such as joint discomfort, tenderness, pain, stiffness or swelling (synovitis) within the past 3-6 months or less. In some instances, a patient having early stage RA has not yet met the ACR/EULAR classification criteria for RA (Aletaha et al., Arth Rheum, 2010, 62(9):2569-2581). In some cases, a patient with early stage RA has not received a diagnosis of RA from a clinician. A patient with early stage RA may experience mild symptoms of RA.

The term “psoriatic arthritis” or “PsA” includes chronic inflammatory arthritic condition that affects the skin, the joints, the insertion sites of tendons, ligaments, and fascia. Psoriatic arthritis is commonly associated with psoriasis. Approximately 6-42% of patients with psoriasis develop psoriatic arthritis. The disease typically appears between the ages of 30-55 but it can be diagnosed during childhood. Men and women have an equal risk for developing the condition. Symptoms of psoriatic arthritis include extra bone formation, joint stiffness, dactylitis, enthesopathy, tendonitis, and spondylitis. Most patients have the classic psoriasis pattern of skin lesions. Scaly, erythematous plaques; guttate lesions, lakes of pus, and erythroderma are psoriatic skin lesions that may be seen in patients with psoriatic arthritis. Nail lesions, including pitting, Beau lines, leukonychia, onycholysis, oil spots, subungual hyperkeratosis, splinter hemorrhages, spotted lunulae, and cracking, are clinical features significantly associated with the development of psoriatic arthritis. Ocular symptoms in psoriatic arthritis include conjunctivitis, iritis, episcleritis, keratoconjunctivitis sicca and aortic insufficiency.

There are about 5 types of psoriatic arthritis: symmetric, asymmetric, distal interphlangeal predominant, spondylitis, and arthritis mutilans. Symmetric PsA accounts for about 50% of the cases and affects joints on both sides of the body at the same time. Asymmetric PsA affects about 35% of the patients with PsA. Inflammation and stiffness at the ends of the fingers and toes are characteristic symptoms of distali interphalangeal predominant. Patients with spondylitis PsA exhibit pain and stiffness in the spine and neck. Arthritis mutilans is a considered the most severe form of PsA and causes deformities in the small joints at the ends of the fingers and toes.

The term “early stage PsA” refers to an initial stage of psoriatic arthritis characterized by the onset of joint symptoms such as joint discomfort, pain, stiffness or swelling within the past 3-6 months or less in patients with psoriasis. In some instances, a patient having early stage PsA has not yet met the CASPAR classification criteria for PsA (Taylor et al., Arth Rheum, 2006, 54:2665-2673). In some cases, a patient with early stage PsA has not received a diagnosis of PsA from a clinician. A patient with early stage PsA may experience mild symptoms of PsA.

The term “rheumatoid factor” or “RF” includes an autoantibody (i.e., an antibody directed against an organism's own tissues) that is typically directed against (i.e., binds to) the Fc (fragment crystallizable) portion of immunoglobulin G (IgG). Rheumatoid factor is most often an IgM autoantibody, but may also be an IgD, IgG, IgA or IgE autoantibody.

The term “anti-citrullinated protein antibody,” “anti-citrullinated peptide antibody,” or “ACPA” includes an autoantibody that specifically targets one or more epitopes in a peptide, polypeptide, or protein sequence where one or more arginine residues have been converted by the enzyme peptidylarginine deiminase into a citrulline residue during a post-translational modification. The presence or level of anti-citrullinated protein antibodies can be detected, determined, or measured using natural or synthetic citrullinated peptides which are immunologically reactive (i.e., immunoreactive) with such antibodies. Non-limiting examples of synthetic citrullinated peptides include cyclic citrullinated peptides (CCP) such as CCP1, which contains a single cyclic citrullinated peptide derived from filaggrin, and/or CCP2, which is a combination of citrullinated peptides selected from screening libraries of citrullinated peptides. Assays for detecting anti-CCP antibodies are available from INOVA Diagnostics, Euro-Diagnostica, Axis-Shield, Phadia, Orgentec Diagostika, and Abbott Diagnostics. Anti-citrullinated protein antibodies are autoantibodies typically associated with rheumatoid arthritis.

The term “subject,” “patient,” or “individual” typically includes humans, but can also include other animals such as, e.g., other primates, rodents, canines, felines, equines, ovines, porcines, and the like.

The term “sample” includes any biological specimen obtained from an individual. Suitable samples for use in the present invention include, without limitation, whole blood, plasma, serum, synovial fluid, saliva, urine, stool, tears, any other bodily fluid, tissue samples (e.g., biopsy), and cellular extracts thereof (e.g., red blood cellular extract). In a preferred embodiment, the sample is a serum sample. The use of samples such as serum, saliva, and urine is well known in the art (see, e.g., Hashida et al., J. Clin. Lab. Anal., 11:267-86 (1997)). One skilled in the art will appreciate that samples such as serum samples can be diluted prior to the analysis of marker levels.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region, such as the promoter and 3′-untranslated region, respectively, as well as intervening sequences (introns) between individual coding segments (exons).

The term “genotype” refers to the genetic composition of an organism, including, for example, whether a diploid organism is heterozygous or homozygous for one or more variant alleles of interest.

The term “polymorphism” refers to the occurrence of two or more genetically determined alternative sequences or alleles in a population. A “polymorphic site” refers to the locus at which divergence occurs. Preferred polymorphic sites have at least two alleles, each occurring at a particular frequency in a population. A polymorphic locus may be as small as one base pair (i.e., single nucleotide polymorphism or SNP). Polymorphic markers include restriction fragment length polymorphisms, variable number of tandem repeats (VNTR's), hypervariable regions, mini satellites, dinucleotide repeats, trinucleotide repeats, tetranucleotide repeats, simple sequence repeats, and insertion elements such as Alu. The first identified allele is arbitrarily designated as the reference allele, and other alleles are designated as alternative alleles, “variant alleles,” or “variances.” The allele occurring most frequently in a selected population is sometimes referred to as the “wild-type” allele or “major” allele. Diploid organisms may be homozygous or heterozygous for the variant alleles. The variant allele may or may not produce an observable physical or biochemical characteristic (“phenotype”) in an individual carrying the variant allele. For example, a variant allele may alter the enzymatic activity of a protein encoded by a gene of interest.

The term “single nucleotide polymorphism” or “SNP” refers to a change of a single nucleotide with a polynucleotide, including within an allele. This can include the replacement of one nucleotide by another, as well as deletion or insertion of a single nucleotide. Single nucleotide polymorphisms may fall within coding sequences of genes, non-coding regions of genes, or in the intergenic regions between genes. SNPs that are not in protein-coding regions may still have consequences for gene splicing, transcription factor binding, or the sequence of non-coding RNA. Most typically, SNPs are biallelic markers, although tri- and tetra-allelic markers can also exist. By way of non-limiting example, a nucleic acid molecule comprising SNP A\C may include an A or C or the complementary alleles thereof at the polymorphic position. For combinations of SNPs, the term “haplotype” is used, e.g. the genotype of the SNPs in a single DNA strand that are linked to one another. In some embodiments, the term “haplotype” can be used to describe a combination of SNP alleles, e.g., the alleles of the SNPs found together on a single DNA molecule. In further embodiments, the SNPs in a haplotype can be in linkage disequilibrium with one another. As there are for genes, there are also bioinformatics databases for SNPs. dbSNP is a SNP database from National Center for Biotechnology Information (NCBI).

The term “risk allele” refers to an allelic variant for a gene that is associated with an increased risk or likelihood of having a specific disease, disorder or condition.

The term “major allele” refers to an allele with the highest frequency at a locus that is observed in a population. For instance, the major allele can be the greater of the two allele frequencies for a single nucleotide polymorphism (SNP).

The term “minor allele” refers to an allele with the lowest frequency at a locus that is observed in a population. For instance, the minor allele can be the lesser of the two allele frequencies for a single nucleotide polymorphism (SNP). There are variations between human populations, so a SNP allele that is common in one geographical or ethnic group may be much rarer, or even absent, in another.

The term “linkage disequilibrium” or “LD” refers to any degree of non-random genetic association between one or more allele(s) of two different polymorphic DNA sequences and that is due to the physical proximity of the two loci. The term can refer to the trend for alleles at nearby loci on haploid genomes to correlate in the population. For example, a and b, alleles at close loci A and B, are said to be in linkage disequilibrium if the a b haplotype (a haplotype is defined as a set of alleles on the same chromosomal segment) has a frequency which is statistically higher than Pₐ×P_{b} (expected frequency if the alleles segregate independently, where Pₐ is the frequency of allele a, and P_{b} that of allele b). LD can be measured using the r² statistic.

The term “cAMP-specific 3′,5′-cyclic phosphodiesterase 4 gene” or “PDE4 gene” refers a gene encoding a cAMP-specific phosphodiesterase 4 polypeptide. The PDE4 gene refers to any one of the PDE4 family genes including the PDE4A gene, the PDE4B gene, the PDE4C gene, the PDE4D gene, and splice variants thereof.

The term “PDE4B gene” refers to the human PDE4B gene located at chromosome 1p31. The genomic sequence of human PDE4B is set forth in, e.g., Gene ID No. 5142 and NC 000001.11 Chromosome 1 Reference GRCh38.p2 Primary Assembly.

The term “PDE4D gene” refers to the human PDE4D gene located at chromosome 5q12. The genomic sequence of human PDE4D is set forth in, e.g., Gene ID No. 5144 and NC_000005.10 Chromosome 5 Reference GRCh38.p2 Primary Assembly.

The term “cAMP-specific 3′,5′-cyclic phosphodiesterase 4 inhibitor,” “phosphodiesterase 4 inhibitor “or “PDE4 inhibitor” refers to a compound or small molecule that inhibits or blocks the activity of the enzyme phosphodiesterase 4 on cAMP.

The term “clinical factor” refers to a physiological attribute that at a certain level may be associated with an increased risk of a specific disease, disorder or condition. Non-limiting examples of clinical factors include patient age, sex, smoking history, family history of the disease, disease duration, disease activity, presence and severity of disease symptoms, genotype, etc.

The term “higher likelihood” or “high likelihood” in the context of a response to treatment refers to an above-average likelihood (chance or probability) that an individual will have a positive response to a treatment.

The term “lower likelihood” or “low likelihood” in the context of a response to treatment refers to an below-average likelihood (chance or probability) that an individual will have a positive response to a treatment.

The term “positive response” with respect to a therapeutic treatment refers to at least a partial marked reduction in the severity, an amelioration of one or more symptoms of a patient's disease or disorder, or slows disease progression.

The term “treat,” “treating” or “treatment” refers to an action that reduces the severity or symptoms of the disease or disorder, or retards or slows the progression or symptoms of the disease or disorder in a patient is suffering from the specified disease or disorder.

The term “therapeutically effective amount or dose” includes a dose of a drug (e.g., a PDE4 inhibitor) that is capable of achieving a therapeutic effect in a subject in need thereof. For example, a therapeutically effective amount of a drug useful for treating RA or PsA can be the amount that is capable of preventing or relieving one or more symptoms associated with RA or PsA. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

### III. Detailed Description of the Embodiments

The present invention provides methods for aiding in the diagnosis of rheumatoid arthritis (RA) versus psoriatic arthritis (PsA) in an individual. Also provided herein are methods for determining whether an individual with RA or an individual with PsA will respond to a PDE4 inhibitor. In addition, provided herein are methods for selecting an individual with RA or an individual with PsA for treatment with a PDE4 inhibitor. Alternatively, the method can be used to identify an individual with RA to receive an anti-TNFα inhibitor therapy. Provided herein are methods for treating an individual with a PDE4 inhibitor if the patient has RA or PsA as well as a specific SNP in the PDE4D gene, a specific SNP in the PDE4B gene or both. The methods disclosed herein can be used to discriminate between PsA and RA patients at the earliest stages of the disease such as before a clinician has made a definitive diagnosis of the patient's disease.

A. Diagnosis of Rheumatoid Arthritis

With rheumatoid arthritis, it is generally believed that many different arthrogenic stimuli activate the immune response in an immunogenetically susceptible individual. Both exogenous infectious agents (e.g., Epstein-Barr virus, rubella virus, cytomegalovirus, herpes virus, human T-cell lymphotropic virus, Mycoplasma, and others) and endogenous proteins such as collagen, proteoglycans, altered immunoglobulins and post-translationally modified proteins like citrullinated proteins have been implicated as a causative agent that triggers an inappropriate host immune response.

Autoimmunity also plays a key role in the progression of the disease. Specifically, a relevant antigen is ingested by antigen-presenting cells (e.g., macrophages or dendritic cells in the synovial membrane), processed, and presented to T lymphocytes. The T cells initiate a cellular immune response and stimulate the proliferation and differentiation of B lymphocytes into plasma cells. The end result is the production of an excessive inappropriate immune response directed against the individual's tissues (e.g., antibodies directed against the Fc portion of autologous IgG, such as rheumatoid factor, and antibodies directed against different citrullinated epitopes, such as anti-CCP autoantibodies). This further amplifies the immune response and hastens the destruction of the cartilage tissue. Once this cascade is initiated numerous mediators of cartilage destruction are responsible for the progression of the condition.

The most commonly used diagnostic criteria for rheumatoid arthritis are those adopted by the American College of Rheumatology (ACR), which are based on a combination of clinical, laboratory and radiological assessments. A patient is classified as having RA if at least four of the following seven criteria are satisfied: (i) morning stiffness lasting at least one hour; (ii) arthritis of three or more joint areas; (iii) arthritis of hand joints; (iv) symmetric arthritis; (v) rheumatoid nodules; (vi) presence of serum rheumatoid factor (RF); and (vii) radiographic changes in hand or wrist joints. Using these criteria, a trained clinician can usually diagnose RA in individuals who have had disease for more than 12 weeks. However, these criteria are largely ineffective for patients during early stages of the disease, such as during the first 12 weeks of disease, during which time irreversible joint damage has already begun. Furthermore, the criteria cannot predict which patients will develop severe erosive disease and those who will benefit from aggressive early disease modifying therapy.

The present invention provides methods for assisting in the diagnosis of RA, such as early stage RA. During the early stages of RA individuals can experience one or more of the following symptoms: one or more mild symptoms, e.g., numbness, tingling, tenderness, pain, warmth of the touch, swelling, redness, or stiffness induced by joint damage in, for example, the small joints of the hands and feet such as the metacarpophalangeal, proximal interphalangeal and metatarsophalangeal joints; fatigue, muscle pain, a low-grade fever and weight loss. In some cases, the individual may experience inflammation of tissues besides the joints, shortness of breath or chest pain. Identification of RA at initial presentation and subsequent initiation of drug treatment can affect the course of the disease, prevent the development of further joint damage, and prevent disease progression. Unfortunately, diagnosis of early RA remains challenging.

1. SNPs in the PDE4B and PDE4D Genes

In some embodiments, the method includes detecting the presence or absence of a specific SNP allele in the PDE4D gene corresponding to rs7733296 and a specific SNP allele in the PDE4B gene corresponding to rs9326069 in a biological sample obtained from the subject. In other embodiments, the method includes detecting the presence or absence of the SNP allele in the PDE4D gene corresponding to rs7733296 or the SNP allele in the PDE4B gene corresponding to rs9326069 in a biological sample obtained from the subject. If the presence of the A allele or a complementary allele thereof (i.e., the T allele) at rs7733296 and the G allele or a complementary allele thereof (i.e., the C allele) at rs9326069 is determined, the subject is predicted to have RA. In other embodiments, the method includes detecting the presence or absence of the SNP in the PDE4D gene corresponding to rs7733296 or the SNP in the PDE4B gene corresponding to rs9326069 in a biological sample obtained from the subject. If the presence of two A alleles or complementary alleles thereof (i.e., two T alleles) at rs7733296 or two G alleles or complementary alleles thereof (i.e., two C alleles) at rs9326069 is determined, the subject is predicted to have RA.

In some embodiments, neighboring SNPs of rs9326069 and/or rs7733296 that are in moderate to high linkage disequilibrium (e.g., r²=0.5, the threshold for moderate LD; r² refers to the square correlation coefficient) to the PDE4B or PDE4D SNP are also detected. In some instances, the presence of one or more neighboring SNPs, e.g., 1, 2, 3, 4, 5, 6, 7, 8, 9, 10 or more neighboring SNPs, of rs9326069 that are in high linkage disequilibrium to rs9326069 are detected. In other instances, the presence of one or more neighboring SNPs, e.g., 1, 2, 3, 4, 5, 6, 7, 8, 9, 10 or more neighboring SNPs, of rs7733296 that are in high linkage disequilibrium to rs7733296 can be detected. Alternatively, one or more (e.g., 1, 2, 3, 4, 5, 6, 7, 8, 9, 10 or more) neighboring SNPs of rs9326069 and one or more (e.g., 1, 2, 3, 4, 5, 6, 7, 8, 9, 10 or more) neighboring SNPs of rs7733296 that are in moderate to high linkage disequilibrium to rs9326069 and rs7733296, respectively are detected. Non-limiting examples of such SNPs are found in Table 1.

TABLE 1
Presence of linkage disequilibrium for
PDE4B (rs9326069) and PDE4D (rs7733296)

| | | Basepair | | | | |
|---|---|---|---|---|---|---|
| | | position | Polymorphism | | | |
| SNP | chr | on chr | ID | Allele 1 | Allele 2 | CEU |
| rs9326069 | 1 | 66217988 | rs10789200 | T | C | 0.86 |
| rs9326069 | 1 | 66218552 | rs10749757 | T | C | 0.52 |
| rs9326069 | 1 | 66224041 | rs4492587 | A | T | 1.00 |
| rs9326069 | 1 | 66224122 | rs6698197 | G | A | 1.00 |
| rs9326069 | 1 | 66225049 | rs75660875 | C | CT | 0.57 |
| rs9326069 | 1 | 66226961 | rs11208733 | T | C | 0.57 |
| rs9326069 | 1 | 66228006 | rs10889582 | T | G | 1.00 |
| rs9326069 | 1 | 66228479 | rs12143459 | G | A | 1.00 |
| rs9326069 | 1 | 66228578 | rs17408644 | T | C | 0.57 |
| rs9326069 | 1 | 66228851 | rs17408658 | A | C | 0.57 |
| rs9326069 | 1 | 66229110 | rs11208735 | A | G | 1.00 |
| rs9326069 | 1 | 66229235 | rs61796987 | G | A | 0.57 |
| rs9326069 | 1 | 66229413 | rs12751974 | A | G | 0.57 |
| rs9326069 | 1 | 66231167 | rs9970005 | C | T | 1.00 |
| rs9326069 | 1 | 66231308 | rs12401401 | G | A | 0.57 |
| rs9326069 | 1 | 66231692 | rs4350183 | G | A | 0.57 |
| rs9326069 | 1 | 66233335 | rs11208738 | C | T | 1.00 |
| rs9326069 | 1 | 66235869 | rs11208740 | G | A | 1.00 |
| rs9326069 | 1 | 66236207 | rs11808116 | C | T | 1.00 |
| rs9326069 | 1 | 66237927 | rs7531141 | G | A | 1.00 |
| rs9326069 | 1 | 66237976 | rs35433628 | TG | T | 0.57 |
| rs9326069 | 1 | 66238890 | rs76758336 | C | T | 0.79 |
| rs9326069 | 1 | 66238907 | rs11208741 | C | T | 0.53 |
| rs9326069 | 1 | 66239762 | rs6696294 | T | C | 0.57 |
| rs9326069 | 1 | 66239897 | rs6659197 | A | C | 1.00 |
| rs9326069 | 1 | 66240228 | rs4655801 | T | G | 0.57 |
| rs9326069 | 1 | 66240846 | rs4557937 | T | C | 1.00 |
| rs9326069 | 1 | 66242820 | rs11208743 | C | T | 0.57 |
| rs9326069 | 1 | 66253599 | rs11208747 | A | G | 0.57 |
| rs9326069 | 1 | 66254084 | rs4385675 | C | T | 1.00 |
| rs9326069 | 1 | 66254868 | rs17449084 | A | T | 0.57 |
| rs9326069 | 1 | 66254949 | rs11208748 | G | T | 0.89 |
| rs9326069 | 1 | 66255034 | rs11208749 | C | T | 0.89 |
| rs9326069 | 1 | 66255225 | rs12034009 | T | C | 0.57 |
| rs9326069 | 1 | 66256227 | rs66534346 | TTC | T | 0.57 |
| rs9326069 | 1 | 66257416 | rs9326069 | G | A | 1.00 |
| rs9326069 | 1 | 66260801 | rs4341342 | T | G | 0.97 |
| rs9326069 | 1 | 66260999 | rs4551569 | T | G | 0.56 |
| rs9326069 | 1 | 66261759 | rs6663176 | A | G | 0.55 |
| rs9326069 | 1 | 66262407 | rs6703532 | T | A | 0.55 |
| rs9326069 | 1 | 66263216 | rs11804553 | G | T | 0.55 |
| rs9326069 | 1 | 66265499 | rs6701146 | G | C | 0.97 |
| rs9326069 | 1 | 66267402 | rs12142070 | C | T | 0.97 |
| rs9326069 | 1 | 66270068 | rs10789202 | C | T | 0.58 |
| rs9326069 | 1 | 66270794 | rs12038590 | T | C | 0.55 |
| rs9326069 | 1 | 66272136 | rs4147194 | C | A | 0.55 |
| rs9326069 | 1 | 66275918 | rs10889585 | T | A | 0.55 |
| rs9326069 | 1 | 66276985 | rs4288570 | T | C | 0.55 |
| rs9326069 | 1 | 66277529 | rs6675417 | C | A | 0.90 |
| rs7733296 | 5 | 58836343 | rs16889948 | T | A | 1.00 |
| rs7733296 | 5 | 58838138 | rs7733296 | T | C | 1.00 |
| rs7733296 | 5 | 58870252 | rs55723056 | T | C | 0.57 |

CEU = Hapmap Caucasian European Samples sequenced in Phase 1 of 1K Genomes Project. The SNP coordinates are provided according to the genome assembly GRCh37/hg 19.

In some embodiments, the method provided herein also includes detecting an allele or a complement thereof of one or more polymorphisms (e.g., SNPs), e.g., 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 polymorphisms, listed in Table 1 to aid in the diagnosis of RA. In some instances, the method can include detecting an allele or a complement thereof of rs9326069 and an allele or a complement thereof of at least one or more SNPs, e.g., 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, or 49 SNPs, listed in Table 1. In some instances, the method can include detecting an allele or a complement thereof of rs7733296 and an allele or a complement thereof of at least one or more SNPs, e.g., 1, 2, or 3 SNPs, listed in Table 1.

In some embodiments, a haplotype as set forth in Table 2 or 3 (haplotypes are represented as columns with the polymorphism designated in the first column) is detected using the method described herein to determine whether a subject has susceptibility to RA. For example, if a T allele or a complement thereof (i.e., an A allele) at rs7733296, a T allele or complement thereof at rs16889948, and a T allele or complement thereof at rs5572356 are detected, it is determined that the subject has susceptibility to RA. In another example, the subject is diagnosed as having RA if the following haplotype is detected, such as an G allele at rs9326069, in addition to one or more of the following SNP alleles: a T allele at rs10789200, a T allele at rs10749757, an A allele at rs4492587, an A allele at rs6698197, a C allele at rs75660875, a T allele at rs11208733, a T allele at rs10889582, a G allele at rs12143459, a T allele at rs17408644, an A allele at rs17408658, an A allele at rs11208735, a G allele at rs61796987, an A allele at rs12751974, a C allele at rs9970005, a G allele at rs12401401, a G allele at rs4350183, a C allele at rs11208738, a G allele at rs11208740, a C allele at rs11808116, a G allele at rs7531141, a TG allele at rs35433628, a C allele at rs76758336, a C allele at rs11208741, a T allele at rs6696294, an A allele at rs6659197, a T allele at rs4655801, a T allele at rs4557937, a C allele at rs11208743, an A allele at rs11208747, a C allele at rs4385675, an A allele at rs17449084, a G allele at rs11208748, a C allele at rs11208749, a T allele at rs12034009, a TTC allele at rs66534346, a G allele at rs9326069, a T allele at rs4341342, a T allele at rs4551569, an A allele at rs6663176, a T allele at rs6703532, a G allele at rs11804553, a G allele at rs6701146, a T allele at rs12142070, a C allele at rs10789202, a T allele at rs12038590, a C allele at rs4147194, a T allele at rs10889585, a T allele at rs4288570, a C allele at rs6675417, or any combination thereof.

TABLE 2
Allele Correlation with PDE4D SNP (rs7733296)

| Frequency | 0.764 | 0.152 | 0.06 | 0.025 |
|---|---|---|---|---|
| rs16889948 | T | A | T | A |
| rs7733296 | T | C | T | C |
| rs55723056 | T | C | C | T |

Frequency in the Caucasian European population.

TABLE 3
Allele Correlation with PDE4B SNP (rs9326069)

| Frequency | 0.606 | 0.135 | 0.074 | 0.067 | 0.021 | 0.014 | 0.012 | 0.012 |
|---|---|---|---|---|---|---|---|---|
| rs10789200 | T | C | C | C | C | C | T | T |
| rs10749757 | T | C | C | T | T | C | T | T |
| rs4492587 | A | T | T | T | T | T | A | T |
| rs6698197 | A | G | G | G | G | G | A | G |
| rs75660875 | C | CT | CT | C | C | CT | C | C |
| rs11208733 | T | C | C | T | T | C | T | T |
| rs10889582 | T | G | G | G | G | G | T | G |
| rs12143459 | G | A | A | A | A | A | G | A |
| rs17408644 | T | C | C | T | T | C | T | T |
| rs17408658 | A | C | C | A | A | C | A | A |
| rs11208735 | A | G | G | G | G | G | A | G |
| rs61796987 | G | A | A | G | G | A | G | G |
| rs12751974 | A | G | G | A | A | G | A | A |
| rs9970005 | C | T | T | T | T | T | C | T |
| rs12401401 | G | A | A | G | G | A | G | G |
| rs4350183 | G | A | A | G | G | A | G | G |
| rs11208738 | C | T | T | T | T | T | C | T |
| rs11208740 | G | A | A | A | A | A | G | A |
| rs11808116 | C | T | T | T | T | T | C | T |
| rs7531141 | G | A | A | A | A | A | G | A |
| rs35433628 | TG | T | T | TG | TG | T | TG | TG |
| rs76758336 | C | T | T | T | C | C | C | T |
| rs11208741 | C | T | T | C | C | T | C | C |
| rs6696294 | T | C | C | T | T | C | T | T |
| rs6659197 | A | C | C | C | C | C | A | C |
| rs4655801 | T | G | G | T | T | G | T | T |
| rs4557937 | T | C | C | C | C | C | T | C |
| rs11208743 | C | T | T | C | C | T | C | C |
| rs11208747 | A | G | G | A | A | G | A | A |
| rs4385675 | C | T | T | T | T | T | C | T |
| rs17449084 | A | T | T | A | A | T | A | A |
| rs11208748 | G | T | T | T | T | T | G | G |
| rs11208749 | C | T | T | T | T | T | C | C |
| rs12034009 | T | C | C | T | T | C | T | T |
| rs66534346 | TTC | T | T | TTC | TTC | T | TTC | TTC |
| rs9326069 | G | A | A | A | A | A | G | A |
| rs4341342 | T | G | G | G | G | G | T | G |
| rs4551569 | T | G | T | G | G | T | T | T |
| rs6663176 | A | G | A | G | G | A | A | A |
| rs6703532 | T | A | T | A | A | T | T | T |
| rs11804553 | G | T | G | T | T | G | G | G |
| rs6701146 | G | C | C | C | C | C | G | C |
| rs12142070 | T | C | C | C | C | C | T | C |
| rs10789202 | C | T | C | T | T | C | C | C |
| rs12038590 | T | C | T | C | C | T | T | T |
| rs4147194 | C | A | C | A | A | C | C | C |
| rs10889585 | T | A | T | A | A | T | T | T |
| rs4288570 | T | C | T | C | C | T | T | T |
| rs6675417 | C | A | A | A | A | A | A | A |

Frequency in the Caucasian European population.

2. Serological Markers

In some embodiments, the presence or level of a serological marker such as rheumatoid factor (RF), an anti-citrullinated protein antibody (ACPA), C-reactive protein (CRP), and combinations thereof is also determined. The presence of one or more (e.g., 1, 2, 3, or more) serological markers can indicate that the subject has RA.

Rheumatoid factors (RFs) are antibodies found in every immunoglobulin subclass (IgE, IgM, IgA and IgG) and directed to the constant Fc-region of immunoglobulins of the IgG subclass. Their presence can be determined by agglutination assays, laser and rate nephelometry, enzyme-linked immunoassays (EIAs) or enzyme linked immunosorbent assays (ELISAs). During the earliest stages of rheumatoid arthritis some patients (about 15-20% of patients) do not have detectable levels of RF. In some cases, patients with detectable RF do not have rheumatoid arthritis.

The antibody response to citrullinated antigens is also a useful diagnostic and prognostic indicator of the presence of RA or of the future progression to RA. Citrullinated antigens (for example, citrullinated filaggrin, citrullinated vimentin, citrullinated fibrinogen, citrullinated lamin B1, citrullinated enolase, citrullinated intermediate filament-derived peptides, viral citrullinated peptides, and fragments thereof) have been shown to be reactive with rheumatoid arthritis autoantibodies in 76% of rheumatoid arthritis sera, with a specificity of 96%. Immunoassays based on the detection of autoantibodies to cyclic citrullinated peptides (anti-cyclic citrullinated peptide autoantibodies; ACPAs) are also useful for the present invention. Examples of such assays include, but are not limited to, the IMMULITE 2000™ anti-CCP IgG assay (Siemens Healthcare Diagnostics, Inc., Tarrytown, N.Y.), the anti-CCP EDIA™ (Euro-Diagnostica, Malmo, Sweden), and the anti-CCP ELISA (Axis-Shield, Dundee, Scotland). Additional assays for the detection of anti-citrullinated peptide antibodies include the first, second, and third generation anti-citrullinated peptides assays, which are also referred to as the anti-CCP1 assay, the anti-CCP2 assay, and the anti-CCP3 assay, respectively.

C-reactive protein (CRP) is a homopentameric Ca²⁺⁻binding acute-phase protein found in the blood in response to inflammation. CRP is typically produced by the liver and adipocytes. CRP production may be induced by IL-6 and indirectly by IL-1. Blood CRP levels in normal, healthy individuals who do not have an infection, tissue injury or inflammatory disorder can be about 5 mg/L or below. Individuals with an inflammatory disorder can have a blood CRP level of about 10 mg/L or more, such as 10 mg/L to about 500 mg/L.

The human CRP polypeptide sequence is set forth in, e.g., Genbank Accession No. NP_000558. The human CRP mRNA (coding) sequence is set forth in, e.g., Genbank Accession No. NM_000567. CRP concentrations can be measured in a patient sample using, e.g., an immunoassay, such as an ELISA, immunoluminometric assay, turbidimetric immunoassay, and particle-enhanced turbidimetric immunoassay. Useful quantitative CRP assays are commercial available from, e.g., Roche Diagnostics and Abbott Laboratories.

In some cases, the presence of one or more serological markers described herein and the presence of one or more SNP described above indicate that the subject has RA. In some embodiments, a subject has RA if the subject has an A allele or a complementary allele thereof at rs7733296 or a G allele or a complementary allele thereof at rs9326069, and also rheumatoid factor, an anti-citrullinated protein antibody, or C-reactive protein. In some embodiments, a subject has RA if the subject has an A allele or a complementary allele thereof at rs7733296 and a G allele or a complementary allele thereof at rs9326069, and also rheumatoid factor, an anti-citrullinated protein antibody, or C-reactive protein. In some cases, the subject has rheumatoid factor and an anti-citrullinated protein antibody, rheumatoid factor and an anti-C-reactive protein, an anti-citrullinated protein antibody and C-reactive protein, or rheumatoid factor, an anti-citrullinated protein antibody, and C-reactive protein.

In some embodiments, the absence of anti-citrullinated protein antibodies (ACPAs) indicates that the subject has PsA. The presence of ACPAs can indicate that the subject has RA. It has been shown that about 75% of RA patients are positive for ACPAs and that ACPA positivity has a specificity of about 90% for RA patients. Thus, there is a subpopulation of ACPA negative subjects who have RA. The methods described herein can be used to determine whether an ACPA negative subject has RA or PsA. In some embodiments, an ACPA negative subject having two A alleles or complementary alleles thereof (i.e., two T alleles) at rs7733296 and/or two G alleles or complementary alleles thereof (i.e., two C alleles) at rs9326069 is predicted to have RA. For instance, a subject that does not have ACPAs and has an AA genotype (or a TT genotype) at rs7733296 and/or a GG genotype (or a CC genotype) at rs9326069 is predicted to be RA positive. In other embodiments, an ACPA negative subject having one or two G alleles or complementary alleles thereof (i.e., one or two C alleles) at rs7733296 and/or one or two A alleles or complementary alleles thereof (i.e., one or two T alleles) at rs9326069 is predicted to have PsA.

3. Clinical Factors

In some embodiments, the presence of a clinical factor, such as the age of the individual, the sex of the individual, smoking history of the individual, a diagnosis of psoriasis, erythrocyte sedimentation rate (ESR), and combinations thereof is also determined. Psoriasis can be diagnosed by a clinician upon physical examination of an individual (e.g., examining the individual's skin, scalp and nails), evaluation the individual's medical history and in some instances, performing a skin biopsy. Types of psoriasis include plaque psoriasis, inverse or flexural psoriasis, erythrodermic psoriasis, pustular psoriasis, and guttate psoriasis. Erythrocyte sedimentation rate (ESR) can be used to measure inflammation in an individual. Elevated ESR such as above average ESR for a normal, healthy subject may be associated in individuals with psoriatic arthritis. In some cases, an ESR greater than 15 mm/h represents an elevated ESR.

In some embodiments, a subject has RA if the subject has an A allele (i.e., one A allele or two A alleles) or a complementary allele thereof at rs7733296 or a G allele (i.e., one G allele or two G alleles) or a complementary allele thereof at rs9326069, and one or more clinical factors described herein. In other embodiments, a subject has RA if the subject has an A allele (i.e., one A allele or two A alleles) and a complementary allele thereof at rs7733296, a G allele (i.e., one G allele or two G alleles) or a complementary allele thereof at rs9326069, and one or more clinical factors described herein.

In some embodiments, the method disclosed herein can include applying a statistical analysis to the presence of the SNP in the PDE4D gene and/or the SNP in the PDE4B gene in combination with the presence of one or more SNP variants of Tables 1-3, the presence or level of the serological marker and/or the presence of the clinical factor. A detailed description of statistical analysis methods is disclosed below.

B. Selecting Treatment for Patients with Rheumatoid Arthritis

Also provided herein is a method for selecting a subject with RA who should receive a treatment with a PDE4 inhibitor. The method includes detecting a SNP in the PDE4D gene corresponding to rs7733296 and/or a SNP in the PDE4B gene corresponding to rs9326069 in a biological sample obtained from the subject. If the subject carries the G allele (i.e., one G allele or two G alleles) or the complementary allele thereof at rs7733296 or the A allele (i.e., one A allele or two A alleles) or the complementary allele thereof at rs9326069, then the subject is selected to receive a treatment containing a PDE4 inhibitor. If the subject carries the G allele (i.e., one G allele or two G alleles) or the complementary allele thereof at rs7733296 and the A allele (i.e., one A allele or two A alleles) or the complementary allele thereof at rs9326069, then the subject is selected to receive a treatment containing a PDE4 inhibitor.

In another aspect, the method described herein is used for determine the likelihood that a subject will positively respond to a PDE4 inhibitor treatment. In some embodiments, a subject having the G allele (i.e., one G allele or two G alleles) or the complementary allele thereof at rs7733296 and/or the A allele (i.e., one A allele or two A alleles) or the complementary allele thereof at rs9326069 is determined to have a higher likelihood of response to a PDE4 inhibitor treatment. In other embodiments, a subject having the G allele (i.e., one G allele or two G alleles) or the complementary allele thereof at rs7733296 and the A allele (i.e., one A allele or two A alleles) or the complementary allele thereof at rs9326069 is determined to have a higher likelihood of response to a PDE4 inhibitor treatment. In some instance, the PDE4 inhibitor is apremilast, cilomilast, diazepam, ibudilast, luteolin, mesembrenone, piclamilast, roflumilast, rolipram, or combinations thereof. In some embodiments, the PDE4 inhibitor treatment is apremilast. In some cases, the subject has been diagnosed with RA.

Also provided herein is a method for treating RA in a subject in need thereof by administering a therapeutically effective amount of a PDE4 inhibitor to a human subject having a G allele (i.e., one G allele or two G alleles) or a complementary allele thereof at rs7733296 or an A allele (i.e., one A allele or two A alleles) or a complementary allele thereof at rs9326069 and suffering from RA. Additionally, provided herein is a method for treating RA in a subject in need thereof by administering a therapeutically effective amount of a PDE4 inhibitor to a human subject having a G allele or a complementary allele thereof at rs7733296 and an A allele or a complementary allele thereof at rs9326069 and suffering from RA. The PDE4 inhibitor can be apremilast, cilomilast, diazepam, ibudilast, luteolin, mesembrenone, piclamilast, roflumilast, rolipram, or combinations thereof. In some cases, the PDE4 inhibitor is apremilast.

C. Diagnosis of Psoriatic Arthritis

There is considerable variation in how clinicians diagnose psoriatic arthritis. Typically, clinicians look for swollen and painful joints, certain patterns of arthritis, and skin and nail changes typical of psoriasis. In addition, blood tests are performed to rule out other types of arthritis and to detect potentially high levels of inflammation and mild anemia. However, the differential diagnosis of PsA remains challenging since many of its features overlap with those of rheumatoid arthritis, reactive inflammatory bowel disease and ankylosing spondylitis. A blood test can be performed to determine if the patient suspected of having PsA has mild anemia and/or an elevated erythrocyte sedimentation rate (ESR or sed rate). ESR provides a basic measure of inflammation. Assays to detect rheumatoid factor (RF) and/or anti-CCP autoantibodies (ACPAs) can be performed to rule out a diagnosis of rheumatoid arthritis. If the patient presents with one or more inflamed, large joints, arthrocentesis can be performed to analyze the joint fluid. Uric acid crystals in such fluid may indicate gout rather than PsA. X-rays can also be performed to identify bony erosions. However, bony erosions resulting from arthritis can be due to RA or PsA. Disclosed herein is a method for diagnosing PsA that is based, in part, on determining a subject's genotype at SNPs within, near or associated with the PDE4D and PDE4B genes.

The present invention provides methods for aiding in the diagnosis of psoriatic arthritis (PsA) in a subject, e.g., a human subject. The method includes detecting the presence of a SNP allele in the PDE4D gene corresponding to rs7733296 and/or the SNP allele in the PDE4B gene corresponding to rs9326069 in a biological sample obtained from the subject. If the presence of the G allele (i.e., one G allele or two G alleles) or the complementary allele thereof at rs7733296 or the A allele (i.e., one A allele or two A alleles) or the complementary allele thereof at rs9326069 is determined, the subject is predicted to have PsA. If the presence of the G allele (i.e., one G allele or two G alleles) or the complementary allele thereof at rs7733296 and the A allele (i.e., one A allele or two A alleles) or the complementary allele thereof at rs9326069 is determined, the subject is predicted to have PsA. For instance, an individual having one or two C alleles at rs7733296 and/or one or two T alleles at rs9326069 may be diagnosed as having PsA.

In some embodiments, the method provided herein also includes detecting an allele or a complement thereof of one or more polymorphisms (e.g., SNPs), e.g., 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 polymorphisms in Table 1 to aid in the diagnosis of PsA. In some instances, the method can include detecting an allele or a complement thereof of rs9326069 and an allele or a complement thereof at least one or more SNP, e.g., 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, or 49 SNPs listed in Table 1. In some instances, the method can include detecting an allele or a complement thereof of rs7733296 and an allele or a complement thereof of at least one or more SNP, e.g., 1, 2, or 3 SNPs listed in Table 1.

In some embodiments, the haplotype as set forth in Table 2 or 3 (haplotypes are represented as columns with the polymorphism designated in the first column) is detected using the method described herein to determine a subject's susceptibility to PsA. For example, if a C allele or a complement thereof (i.e., a G allele) at rs7733296, an A allele or complement thereof at rs16889948, and a C allele or complement thereof at rs5572356 are detected, it is determined that the subject has susceptibility to PsA. In another example, the subject is diagnosed as having PsA if the following haplotype is detected, such as an A allele at rs9326069, in addition to one or more of the following SNP alleles: a C allele at rs10789200, a C allele at rs10749757, a T allele at rs4492587, a G allele at rs6698197, a CT allele at rs75660875, a C allele at rs11208733, a G allele at rs10889582, an A allele at rs12143459, a C allele at rs17408644, a C allele at rs17408658, a G allele at rs11208735, an A allele at rs61796987, a G allele at rs12751974, a T allele at rs9970005, an A allele at rs12401401, an A allele at rs4350183, a T allele at rs11208738, an A allele at rs11208740, a T allele at rs11808116, an A allele at rs7531141, a T allele at rs35433628, a T allele at rs76758336, a T allele at rs11208741, a C allele at rs6696294, a C allele at rs6659197, a G allele at rs4655801, a C allele at rs4557937, a T allele at rs11208743, a G allele at rs11208747, a T allele at rs4385675, a T allele at rs17449084, a T allele at rs11208748, a T allele at rs11208749, a C allele at rs12034009, a T allele at rs66534346, an A allele at rs9326069, a G allele at rs4341342, a G allele at rs4551569, a G allele at rs6663176, an A allele at rs6703532, a T allele at rs11804553, a C allele at rs6701146, a C allele at rs12142070, a T allele at rs10789202, a C allele at rs12038590, an A allele at rs4147194, an A allele at rs10889585, a C allele at rs4288570, an A allele at rs6675417, or any combination thereof.

In some embodiments, the presence or level of a serological marker as described above, such as rheumatoid factor (RF), an anti-citrullinated protein antibody (ACPA), C-reactive protein (CRP), and combinations thereof is also determined. The presence of one or more serological markers can indicate that the subject does not have PsA. For instance, a subject does not have PsA if the subject has rheumatoid factor; an anti-citrullinated protein antibody; C-reactive protein; or either rheumatoid factor and an anti-citrullinated protein antibody; rheumatoid factor and an anti-C-reactive protein; an anti-citrullinated protein antibody and C-reactive protein; or rheumatoid factor, an anti-citrullinated protein antibody, and C-reactive protein. In addition, the absence of a G allele or the complementary allele thereof at rs7733296 or an A allele or the complementary allele thereof at rs9326069 as well as the presence of one or more serological markers described herein rules out PsA.

In some embodiments, the presence or status of a clinical factor, such as the age of the individual, the sex of the individual, smoking history of the individual, a diagnosis of psoriasis, erythrocyte sedimentation rate (ESR), and combinations thereof is also determined. A clinician can diagnose psoriasis by taking a medical history and examining the subject's skin, scalp and nails. If a diagnosis of psoriasis is made, the subject may also be diagnosed as having psoriatic arthritis.

In some embodiments, the method described herein also includes applying a statistical analysis to the presence of the SNP in the PDE4D gene and/or the SNP in the PDE4B gene in combination with the presence of one or more SNP variants listed in Tables 1-3, the presence or level of one or more serological markers and/or the presence or status of one or more clinical factor. Descriptions of useful statistical analysis methods are described below.

D. Selecting Treatment for Patients with Psoriatic Arthritis

Provided herein is also a method for selecting a patient with PsA for treatment with a PDE4 inhibitor, such as apremilast, cilomilast, diazepam, ibudilast, luteolin, mesembrenone, piclamilast, roflumilast, rolipram, and combinations thereof. The method includes detecting the presence of a SNP in a PDE4 gene, such as the SNP in the PDE4D gene at rs7733296 and/or the SNP in the PDE4B gene at rs9326069 in a sample from the patient. If the patient carries a G allele (i.e., one G allele or two G alleles) or the complementary allele thereof at rs7733296 and/or an A allele (i.e., one A allele or two A alleles) or the complementary allele thereof at rs9326069, then the patient is selected to receive a PDE4 inhibitor. In preferred embodiments, the patient is selected to receive apremilast. In some embodiments, the method also includes detecting the presence of one or more SNP alleles set forth in Tables 1-3. In other embodiments, the presence of one or more SNP alleles in Tables 1-3 are detected instead of the SNP alleles at rs7733296 and/or rs9326069.

In some embodiments, the method also includes detecting the presence or absence of one or more serological markers described herein and/or the presence or absence of one or more clinical factors described herein. In some embodiments, the method further includes applying a statistical analysis to the presence of the SNP allele(s) in the PDE4 gene in combination with the presence of one or more SNP alleles listed in Tables 1-3, the presence or level of the serological marker and/or the presence of the clinical factor. The statistical analysis can improve the sensitivity, specificity, and/or overall accuracy of therapy selection with the PDE4 inhibitor.

Also, provided herein is a method for determining that a patient with PsA will respond to a PDE4 inhibitor treatment. The patient may be diagnosed with PsA or experiencing symptoms of PsA. The method includes determining the presence of a SNP in a PDE4 gene, such as a SNP in the PDE4D gene, a SNP in the PDE4B gene, or a combination thereof in a sample taken from the patient. The SNP in the PDE4D gene can correspond to rs7733296. The SNP in the PDE4B gene can correspond to rs9326069. If it is determined that the patient has the G allele (i.e., one G allele or two G alleles) or the complementary allele thereof at rs7733296 and/or the A allele (i.e., one A allele or two A alleles) or the complementary allele thereof at rs9326069, then the patient has a higher likelihood of positively responding to a PDE4 inhibitor, such as apremilast, cilomilast, diazepam, ibudilast, luteolin, mesembrenone, piclamilast, roflumilast, rolipram, and combinations thereof. In some embodiments, the presence of a G allele (i.e., one G allele or two G alleles) or the complementary allele thereof at rs7733296 and/or an A allele (i.e., one A allele or two A alleles) or the complementary allele thereof at rs9326069 indicates that the patient with PsA will respond to and should be administered apremilast.

Also provided herein is a method for treating PsA in a subject in need thereof by administering a therapeutically effective amount of a PDE4 inhibitor to a human subject having a G allele (i.e., one G allele or two G alleles) or a complementary allele thereof at rs7733296 or an A allele (i.e., one A allele or two A alleles) or a complementary allele thereof at rs9326069 and suffering from PsA. Also provided herein is a method for treating PsA in a subject in need thereof by administering a therapeutically effective amount of a PDE4 inhibitor to a human subject having a G allele (i.e., one G allele or two G alleles) or a complementary allele thereof at rs7733296 and an A allele (i.e., one A allele or two A alleles) or a complementary allele thereof at rs9326069 and suffering from PsA. The PDE4 inhibitor can be apremilast, cilomilast, diazepam, ibudilast, luteolin, mesembrenone, piclamilast, roflumilast, rolipram, or combinations thereof.

E. Methods of Genotyping

A variety of means can be used to genotype an individual at a polymorphic site in a gene or any other genetic marker described herein to determine whether a sample (e.g., a nucleic acid sample) contains a specific variant allele or haplotype. For example, enzymatic amplification of nucleic acid from an individual can be conveniently used to obtain nucleic acid for subsequent analysis. The presence of a specific variant allele or haplotype in one or more genetic markers of interest can also be determined directly from the individual's nucleic acid without enzymatic amplification. In some embodiments, an individual is genotyped at the PDE4D locus. In other embodiments, an individual is genotyped at the PDE4B locus. In yet other embodiments, an individual is genotype at both the PDE4D and PDE4B loci.

Genotyping of nucleic acid from an individual, whether amplified or not, can be performed using any of various techniques. Useful techniques include, without limitation, polymerase chain reaction (PCR) based analysis, sequence analysis, and electrophoretic analysis, which can be used alone or in combination. As used herein, the term “nucleic acid” means a polynucleotide such as a single- or double-stranded DNA or RNA molecule including, for example, genomic DNA, cDNA and mRNA. This term encompasses nucleic acid molecules of both natural and synthetic origin as well as molecules of linear, circular, or branched configuration representing either the sense or antisense strand, or both, of a native nucleic acid molecule. It is understood that such nucleic acids can be unpurified, purified, or attached, for example, to a synthetic material such as a bead or column matrix.

Material containing nucleic acid is routinely obtained from individuals. Such material is any biological matter from which nucleic acid can be prepared or extracted. As non-limiting examples, material can be whole blood, serum, plasma, synovial fluid, saliva, cheek swab, sputum, or other bodily fluid or tissue that contains nucleic acid. In one embodiment, a method of the present invention is practiced with whole blood, which can be obtained readily by non-invasive or minimally invasive means and used to prepare genomic DNA. In another embodiment, genotyping involves amplification of an individual's nucleic acid using the polymerase chain reaction (PCR). Use of PCR for the amplification of nucleic acids is well known in the art (see, e.g., Mullis et al. (Eds.), The Polymerase Chain Reaction, Birkhäuser, Boston, (1994)). In yet another embodiment, PCR amplification is performed using one or more fluorescently labeled primers. In a further embodiment, PCR amplification is performed using one or more labeled or unlabeled primers that contain a DNA minor groove binder.

Any of a variety of different primers can be used to amplify an individual's nucleic acid by PCR in order to determine the presence of a variant allele in one or more genes or other genetic marker in a method of the invention. Non-limiting examples of genes include PDE4D and PDE4B. For example, the PCR primers can be used to amplify specific regions of the PDE4D or PDE4B locus. As understood by one skilled in the art, additional primers for PCR analysis can be designed based on the sequence flanking the polymorphic site(s) of interest in the PDE4D or PDE4B gene or other genetic marker (e.g., SNP variants of Table 1). As a non-limiting example, a sequence primer can contain from about 15 to about 30 nucleotides of a sequence upstream or downstream of the polymorphic site of interest in the PDE4D or PDE4B gene or other genetic marker. Such primers generally are designed to have sufficient guanine and cytosine content to attain a high melting temperature which allows for a stable annealing step in the amplification reaction. Several computer programs, such as Primer Select, are available to aid in the design of PCR primers.

A Taqman® allelic discrimination assay available from Applied Biosystems can be useful for genotyping an individual at a polymorphic site and thereby determining the presence of a particular variant allele or haplotype in one or more of the PDE4D or PDE4B genes, or other genetic marker (e.g., SNP variants of Table 1). In a Taqman® allelic discrimination assay, a specific fluorescent dye-labeled probe for each allele is constructed. The probes contain different fluorescent reporter dyes such as FAM and VIC to differentiate amplification of each allele. In addition, each probe has a quencher dye at one end which quenches fluorescence by fluorescence resonance energy transfer. During PCR, each probe anneals specifically to complementary sequences in the nucleic acid from the individual. The 5′ nuclease activity of Taq polymerase is used to cleave only probe that hybridizes to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Those skilled in the art understand that improved specificity in allelic discrimination assays can be achieved by conjugating a DNA minor groove binder (MGB) group to a DNA probe as described, e.g., in Kutyavin et al., Nuc. Acids Research 28:655-661 (2000). Minor groove binders include, but are not limited to, compounds such as dihydrocyclopyrroloindole tripeptide (DPI3).

Sequence analysis can also be useful for genotyping an individual according to the methods described herein to determine the presence of a particular variant allele or haplotype in the PDE4D or PDE4B gene or other genetic marker (e.g., SNP variants of Table 1). As is known by those skilled in the art, a variant allele of interest can be detected by sequence analysis using the appropriate primers, which are designed based on the sequence flanking the polymorphic site of interest in one or more of the PDE4D or PDE4B genes, or another genetic marker. Additional or alternative sequence primers can contain from about 15 to about 30 nucleotides of a sequence that corresponds to a sequence about 40 to about 400 base pairs upstream or downstream of the polymorphic site of interest in one or more of the PDE4D or PDE4B genes, or another genetic marker. Such primers are generally designed to have sufficient guanine and cytosine content to attain a high melting temperature which allows for a stable annealing step in the sequencing reaction.

The term “sequence analysis” includes any manual or automated process by which the order of nucleotides in a nucleic acid is determined. As an example, sequence analysis can be used to determine the nucleotide sequence of a sample of DNA. The term sequence analysis encompasses, without limitation, chemical and enzymatic methods such as dideoxy enzymatic methods including, for example, Maxam-Gilbert and Sanger sequencing as well as variations thereof. The term sequence analysis further encompasses, but is not limited to, capillary array DNA sequencing, which relies on capillary electrophoresis and laser-induced fluorescence detection and can be performed using instruments such as the MegaBACE 1000 or ABI 3700. As additional non-limiting examples, the term sequence analysis encompasses thermal cycle sequencing (see, Sears et al., Biotechniques 13:626-633 (1992)); solid-phase sequencing (see, Zimmerman et al., Methods Mol. Cell Biol. 3:39-42 (1992); and sequencing with mass spectrometry, such as matrix-assisted laser desorption/ionization time-of-flight mass spectrometry (see, MALDI-TOF MS; Fu et al., Nature Biotech. 16:381-384 (1998)). The term sequence analysis further includes, but is not limited to, sequencing by hybridization (SBH), which relies on an array of all possible short oligonucleotides to identify a segment of sequence (see, Chee et al., Science 274:610-614 (1996); Drmanac et al., Science 260:1649-1652 (1993); and Drmanac et al., Nature Biotech. 16:54-58 (1998)). One skilled in the art understands that these and additional variations are encompassed by the term sequence analysis as defined herein.

Electrophoretic analysis also can be useful in genotyping an individual according to the methods of the present invention to determine the presence of a particular variant allele or haplotype in one or more of the PDE4D or PDE4B genes, or another genetic marker. “Electrophoretic analysis” as used herein in reference to one or more nucleic acids such as amplified fragments includes a process whereby charged molecules are moved through a stationary medium under the influence of an electric field. Electrophoretic migration separates nucleic acids primarily on the basis of their charge, which is in proportion to their size, with smaller molecules migrating more quickly. The term electrophoretic analysis includes, without limitation, analysis using slab gel electrophoresis, such as agarose or polyacrylamide gel electrophoresis, or capillary electrophoresis. Capillary electrophoretic analysis generally occurs inside a small-diameter (50-100 m) quartz capillary in the presence of high (kilovolt-level) separating voltages with separation times of a few minutes. Using capillary electrophoretic analysis, nucleic acids are conveniently detected by UV absorption or fluorescent labeling, and single-base resolution can be obtained on fragments up to several hundred base pairs. Such methods of electrophoretic analysis, and variations thereof, are well known in the art, as described, for example, in Ausubel et al., Current Protocols in Molecular Biology Chapter 2 (Supplement 45) John Wiley & Sons, Inc. New York (1999).

Restriction fragment length polymorphism (RFLP) analysis can also be useful for genotyping an individual according to the methods of the present invention to determine the presence of a particular variant allele or haplotype in the PDE4D or PDE4B gene or other genetic marker (see, Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, “restriction fragment length polymorphism analysis” includes any method for distinguishing polymorphic alleles using a restriction enzyme, which is an endonuclease that catalyzes degradation of nucleic acid following recognition of a specific base sequence, generally a palindrome or inverted repeat. One skilled in the art understands that the use of RFLP analysis depends upon an enzyme that can differentiate a variant allele from a wild-type or other allele at a polymorphic site.

In addition, allele-specific oligonucleotide hybridization can be useful for genotyping an individual in the methods described herein to determine the presence of a particular variant allele or haplotype in one or more of the PDE4D or PDE4B genes, or another genetic marker (e.g., SNP variant in Table 1). Allele-specific oligonucleotide hybridization is based on the use of a labeled oligonucleotide probe having a sequence perfectly complementary, for example, to the sequence encompassing the variant allele. Under appropriate conditions, the variant allele-specific probe hybridizes to a nucleic acid containing the variant allele but does not hybridize to the one or more other alleles, which have one or more nucleotide mismatches as compared to the probe. If desired, a second allele-specific oligonucleotide probe that matches an alternate (e.g., wild-type) allele can also be used. Similarly, the technique of allele-specific oligonucleotide amplification can be used to selectively amplify, for example, a variant allele by using an allele-specific oligonucleotide primer that is perfectly complementary to the nucleotide sequence of the variant allele but which has one or more mismatches as compared to other alleles (Mullis et al., supra). One skilled in the art understands that the one or more nucleotide mismatches that distinguish between the variant allele and other alleles are often located in the center of an allele-specific oligonucleotide primer to be used in the allele-specific oligonucleotide hybridization. In contrast, an allele-specific oligonucleotide primer to be used in PCR amplification generally contains the one or more nucleotide mismatches that distinguish between the variant and other alleles at the 3′ end of the primer.

A heteroduplex mobility assay (HMA) is another well-known assay that can be used for genotyping in the methods of the present invention to determine the presence of a particular variant allele or haplotype in one or more of the PDE4D or PDE4B genes, or another genetic marker. HMA is useful for detecting the presence of a variant allele since a DNA duplex carrying a mismatch has reduced mobility in a polyacrylamide gel compared to the mobility of a perfectly base-paired duplex (see, Delwart et al., Science, 262:1257-1261 (1993); White et al., Genomics, 12:301-306 (1992)).

The technique of single strand conformational polymorphism (SSCP) can also be useful for genotyping in the methods described herein to determine the presence of a particular variant allele or haplotype in one or more of the PDE4D or PDE4B genes, or another genetic marker (see, Hayashi, Methods Applic., 1:34-38 (1991)). This technique is used to detect variant alleles based on differences in the secondary structure of single-stranded DNA that produce an altered electrophoretic mobility upon non-denaturing gel electrophoresis. Variant alleles are detected by comparison of the electrophoretic pattern of the test fragment to corresponding standard fragments containing known alleles.

Denaturing gradient gel electrophoresis (DGGE) can also be useful in the methods of the invention to determine the presence of a particular variant allele or haplotype in one or more of the PDE4D or PDE4B genes, or another genetic marker. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (see, Sheffield et al., “Identifying DNA Polymorphisms by Denaturing Gradient Gel Electrophoresis” in Innis et al., supra, 1990).

Other molecular methods useful for genotyping an individual are known in the art and useful in the methods of the present invention. Such well-known genotyping approaches include, without limitation, automated sequencing, next-generation sequencing and RNase mismatch techniques (see, Winter et al., Proc. Natl. Acad. Sci., 82:7575-7579 (1985)). Furthermore, one skilled in the art understands that, where the presence of multiple variant alleles is to be determined, individual variant alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple variant alleles can be detected in individual reactions or in a single reaction (a “multiplex” assay).

In view of the above, one skilled in the art realizes that the methods of the present invention for providing diagnostic information to improve the detection of early stages of the disease, where different types of arthritis such as rheumatoid arthritis (RA) and psoriatic arthritis (PsA) are less differentiated and more difficult to diagnose, for providing prognostic and predictive information regarding the outcome and course of progression of RA and PsA, for providing information regarding the likelihood of response to treatment with PDE4 inhibitors in patients with RA or PsA, and for providing information regarding the selection of a suitable therapeutic regimen for the treatment of RA or PsA (e.g., by determining the presence of one or more variant alleles of genes such as, but not limited to, PDE4D and/or PDE4B) can be practiced using one or any combination of the well-known genotyping assays described above or other assays known in the art.

F. Statistical Analysis

In some aspects, the present invention provides methods for differentiating between RA and PsA (e.g., for improving the detection of early stages of the disease) and for determining the likelihood of response to treatment with PDE4 inhibitors by detecting the presence of one or more variant alleles (e.g., SNPs) at the PDE4B locus and/or PDE4D locus, alone or in combination with detecting the presence of one or more SNPs in linkage disequilibrium with the SNP of the PDE4B locus and/or the PDE4D locus, the presence or level of one or more other biomarkers (e.g., RF, anti-CCP antibodies, etc.) and/or the presence of one or more clinical factors (e.g., patient age, sex, etc.), and applying a statistical analysis such as quantile analysis or a learning statistical classifier system to the genotype(s) detected at the PDE4D locus and/or PDE4B locus and optionally to the presence of one or more SNP variants, the presence or level of the one or more other biomarkers and/or to the presence of one or more clinical factors. In certain embodiments, the use of statistical analyses in the methods of the present invention advantageously provide improved sensitivity, specificity, negative predictive value, positive predictive value, and/or overall accuracy for the early diagnosis of RA or PsA, for differentiating between RA versus PsA, and for predicting or identifying the probability that a patient with RA or PsA will respond to treatment with one or more PDE4 inhibitors.

The term “statistical analysis” or “statistical algorithm” or “statistical process” includes any of a variety of statistical methods and models used to determine relationships between variables. In the present invention, the variables are the presence, level, or genotype of at least one marker of interest. Any number of markers can be analyzed using a statistical analysis described herein. For example, the presence or level of 1, 2, 3, 4, 5, 6, 7, 8, 9, 10, 11, 12, 13, 14, 15, 16, 17, 18, 19, 20, 25, 30, 35, 40, 45, 50, 55, 60, or more markers can be included in a statistical analysis. In one embodiment, logistic regression is used. In another embodiment, linear regression is used. In certain preferred embodiments, the statistical analyses comprise a quantile measurement of one or more markers, e.g., within a given population, as a variable. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present invention can also include the use of percentile ranges of marker levels (e.g., tertiles, quartile, quintiles, etc.), or their cumulative indices (e.g., quartile sums of marker levels to obtain quartile sum scores (QSS), etc.) as variables in the statistical analyses (just as with continuous variables).

In some embodiments, the statistical analyses comprise one or more learning statistical classifier systems. As used herein, the term “learning statistical classifier system” includes a machine learning algorithmic technique capable of adapting to complex data sets (e.g., panel of markers of interest) and making decisions based upon such data sets. In some embodiments, a single learning statistical classifier system such as a decision/classification tree (e.g., random forest (RF) or classification and regression tree (CART)) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (CART), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as nave learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

Random forests are learning statistical classifier systems that are constructed using an algorithm developed by Leo Breiman and Adele Cutler. Random forests use a large number of individual decision trees and decide the class by choosing the mode (i.e., most frequently occurring) of the classes as determined by the individual trees. Random forest analysis can be performed, e.g., using the RandomForests software available from Salford Systems (San Diego, Calif.). See, e.g., Breiman, Machine Learning, 45:5-32 (2001); and at website stat-www.berkeley.edu/users/breiman/RandomForests/cc_home.htm, for a description of random forests.

Classification and regression trees represent a computer intensive alternative to fitting classical regression models and are typically used to determine the best possible model for a categorical or continuous response of interest based upon one or more predictors. Classification and regression tree analysis can be performed, e.g., using the CART software available from Salford Systems or the Statistica data analysis software available from StatSoft, Inc. (Tulsa, Okla.). A description of classification and regression trees is found, e.g., in Breiman et al. “Classification and Regression Trees,” Chapman and Hall, New York (1984); and Steinberg et al., “CART: Tree-Structured Non-Parametric Data Analysis,” Salford Systems, San Diego, (1995).

Neural networks are interconnected groups of artificial neurons that use a mathematical or computational model for information processing based on a connectionist approach to computation. Typically, neural networks are adaptive systems that change their structure based on external or internal information that flows through the network. Specific examples of neural networks include feed-forward neural networks such as perceptrons, single-layer perceptrons, multi-layer perceptrons, backpropagation networks, ADALINE networks, MADALINE networks, Learnmatrix networks, radial basis function (RBF) networks, and self-organizing maps or Kohonen self-organizing networks; recurrent neural networks such as simple recurrent networks and Hopfield networks; stochastic neural networks such as Boltzmann machines; modular neural networks such as committee of machines and associative neural networks; and other types of networks such as instantaneously trained neural networks, spiking neural networks, dynamic neural networks, and cascading neural networks. Neural network analysis can be performed, e.g., using the Statistica data analysis software available from StatSoft, Inc. See, e.g., Freeman et al., In “Neural Networks: Algorithms, Applications and Programming Techniques,” Addison-Wesley Publishing Company (1991); Zadeh, Information and Control, 8:338-353 (1965); Zadeh, “IEEE Trans. on Systems, Man and Cybernetics,” 3:28-44 (1973); Gersho et al., In “Vector Quantization and Signal Compression,” Kluywer Academic Publishers, Boston, Dordrecht, London (1992); and Hassoun, “Fundamentals of Artificial Neural Networks,” MIT Press, Cambridge, Mass., London (1995), for a description of neural networks.

Support vector machines are a set of related supervised learning techniques used for classification and regression and are described, e.g., in Cristianini et al., “An Introduction to Support Vector Machines and Other Kernel-Based Learning Methods,” Cambridge University Press (2000). Support vector machine analysis can be performed, e.g., using the SVM^{light} software developed by Thorsten Joachims (Cornell University) or using the LIBSVM software developed by Chih-Chung Chang and Chih-Jen Lin (National Taiwan University).

As used herein, the term “sensitivity” refers to the probability that a diagnostic, prognostic, or predictive method of the present invention gives a positive result when the sample is positive, e.g., having the predicted diagnosis of RA or PsA, or having the predicted differentiation between RA and PsA. Sensitivity is calculated as the number of true positive results divided by the sum of the true positives and false negatives. Sensitivity essentially is a measure of how well the present invention correctly identifies those who have the predicted diagnosis of RA, the predicted diagnosis of PsA, or the predicted differentiation between RA and PsA from those who do not have the predicted diagnosis of RA, the predicted diagnosis of PsA, or the predicted differentiation between RA and PsA. The statistical methods and models can be selected such that the sensitivity is at least about 60%, and can be, e.g., at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “specificity” refers to the probability that a diagnostic, prognostic, or predictive method of the present invention gives a negative result when the sample is not positive, e.g., not having the predicted diagnosis of RA or PsA, or not having the predicted differentiation between RA and PsA. Specificity is calculated as the number of true negative results divided by the sum of the true negatives and false positives. Specificity essentially is a measure of how well the present invention excludes those who do not have the predicted diagnosis of RA, the predicted diagnosis of PsA, or the predicted differentiation between RA and PsA from those who do have the predicted diagnosis of RA, the predicted diagnosis of PsA, or the predicted differentiation between RA and PsA. The statistical methods and models can be selected such that the specificity is at least about 60%, and can be, e.g., at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

As used herein, the term “negative predictive value” or “NPV” refers to the probability that an individual identified as not having the predicted diagnosis of RA or PsA, or not having the predicted differentiation between RA and PsA, actually does not have the predicted diagnosis of RA, the predicted diagnosis of PsA, or the predicted differentiation between the RA and PsA. Negative predictive value can be calculated as the number of true negatives divided by the sum of the true negatives and false negatives. Negative predictive value is determined by the characteristics of the diagnostic or prognostic method as well as the prevalence of the disease in the population analyzed. The statistical methods and models can be selected such that the negative predictive value in a population having a disease prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “positive predictive value” or “PPV” refers to the probability that an individual identified as having the predicted diagnosis of RA or PsA, or having the predicted differentiation between RA and PsA, actually has the predicted diagnosis of RA, the predicted diagnosis of PsA, or the predicted differentiation between RA and PsA. Positive predictive value can be calculated as the number of true positives divided by the sum of the true positives and false positives. Positive predictive value is determined by the characteristics of the diagnostic or prognostic method as well as the prevalence of the disease in the population analyzed. The statistical methods and models can be selected such that the positive predictive value in a population having a disease prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Predictive values, including negative and positive predictive values, are influenced by the prevalence of the disease in the population analyzed. In the present invention, the statistical methods and models can be selected to produce a desired clinical parameter for a clinical population with a particular RA or PsA prevalence. For example, statistical methods and models can be selected for an RA or PsA prevalence of up to about 1%, 2%, 3%, 4%, 5%, 6%, 7%, 8%, 9%, 10%, 15%, 20%, 25%, 30%, 35%, 40%, 45%, 50%, 55%, 60%, 65%, or 70%, which can be seen, e.g., in a clinician's office such as a rheumatologist's office or a general practitioner's office.

As used herein, the term “overall agreement” or “overall accuracy” refers to the accuracy with which a method of the present invention diagnoses RA, diagnoses PsA, or differentiates between RA and PsA. Overall accuracy is calculated as the sum of the true positives and true negatives divided by the total number of sample results and is affected by the prevalence of the disease in the population analyzed. For example, the statistical methods and models can be selected such that the overall accuracy in a patient population having a disease prevalence is at least about 40%, and can be, e.g., at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

G. PDE4 Inhibitors

Phosphodiesterases (PDEs) play an important role in various biological processes by hydrolyzing the key second messengers adenosine and guanosine 3′,5′-cyclic monophosphates (cAMP and cGMP, respectively) into their corresponding 5′-monophosphate nucleotides. Therefore, inhibition of PDE activity produces an increase of cAMP and cGMP intracellular levels that activate specific protein phosphorylation pathways involved in a variety of functional responses. At least 11 families of PDEs exist, some of which (PDE 3, 4, 7, 8) are specific for cAMP, and others (PDE 5, 6, 9) for cGMP, while other family members have dual specificity (PDE 1, 2, 10, 11). PDEs are expressed in a tissue and cell specific manner, and expression also changes depending on the cell state. For example, resting T lymphocytes express mainly PDE3 and PDE4.

The four PDE4 subfamilies are encoded by separate genes (A, B, C, D) that generate many isoforms through the use of alternative mRNA splicing and distinct promoters. Isoforms generated by the four PDE4 subfamilies are each individually characterized by unique N-terminal regions. They can be divided into long forms, which possess both the Upstream Conserved Region 1 (UCR1) and Upstream Conserved Region (UCR2) regulatory regions, while the short isoforms lack UCR1 and the super-short isoforms lack UCR1 and also have a truncated UCR2.

PDE4 inhibitors have been developed for the treatment of inflammatory airways disease, asthma, chronic obstructive pulmonary disease (COPD), allergic rhinitis, psoriasis, rheumatoid arthritis, depression, schizophrenia, Alzheimer's disease, memory loss, cancer, dermatitis and multiple sclerosis. Inhibition of PDE4 has been associated with an anti-inflammatory response associated with T cells as well as monocytes, macrophages, mast cells, basophils and neutrophils. To date, the majority of PDE4 selective inhibitors serve to inhibit PDE4 isoforms from all the four subfamilies (e.g., PDE4A, PDE4B, PDE4C, and PDE4D) with either little or no PDE4 subfamily selectivity. Useful PDE4 inhibitors for the present invention include, but are not limited to, apremilast, cilomilast, diazepam, ibudilast, luteolin, mesembrenone, piclamilast, roflumilast, rolipram, revamilast, ciclamilast, AN2728, derivatives thereof, and combinations thereof. Additional PDE4 inhibitors include but at not limited to those described in, for example, International Patent Publication Nos. WO2003080049, WO2003070279, WO2012019251, WO2013084182 and WO2013101810.

In some embodiments, a PDE4 inhibitor therapy is administered alone or in combination with another therapy such as, but not limited to a biologic anti-TNFα inhibitorsuch as etanercept (ENBREL™), adalimumab (HUMIRA™), infliximab (REMICADE™), golimumab (SIMPONI®) and certolizumab pegol (CIMZIA®), ESBA105, and pegsunercept, a biologic IL-17 inhibitor such as secukinumab, brodalumab, and ixekizumab, an anti-IL 12/23 inhibitor such as usetekinumab and aplimod, a non-steroidal anti-inflammatory drug (NSAID), methotrexate, sulfasalazine, leflunomide, cyclosporine A, a JAK inhibitor such as tofacitinib, LY3009104, VX-509 and ASP015K, a protein kinase C inhibitor such as sotrastaurin, BMS-582949, azathioprine (IMURAN®), sulfasalazine, retinoid, corticosteroid, Cox-2 inhibitor, and the like.

### IV. Example

The present invention will be described in greater detail by way of a specific example. The following example is offered for illustrative purposes, and is not intended to limit the invention in any manner. Those of skill in the art will readily recognize a variety of noncritical parameters which can be changed or modified to yield essentially the same results.

### Example 1. Identification of Genetic Variations Differentially Associated with Psoriatic Arthritis (PsA) Versus Rheumatoid Arthritis (RA)

This example provides the results of a genome-wide association study (GWAS) of well-characterized patients with either PsA or RA. Over 500,000 markers were genotyped, thereby capturing the most common genetic variations. In particular, 550,000 single nucleotide polymorphisms (SNPs) and 4,000 copy number polymorphisms (CNPs) associated with disease specific pathways were analyzed in 950 PsA samples and 1,201 RA samples. The patient samples and corresponding clinical data were collected from over 30 clinical departments across Spain participating in the IMID consortium. A similar method was used previously to identify new risk loci for chronic inflammatory diseases including KLF12 for RA as well as EP300 and IFNGR2 for Crohn's disease.

One SNP associated with a new locus (PDE4D) was identified; rs7733296 (intronic) demonstrated a p-value of 2e⁻⁶. The SNP was validated in an independent cohort of 693 PsA versus 1,679 RA samples. The SNP was directly genotyped in the independent dataset and was shown to be associated with a RA vs. PsA phenotype (p<0.01). The minor allele (i.e., a G allele) or the complementary allele thereof at rs7733296 was more frequent in PsA patients. The major allele (i.e., an A allele) or the complementary allele thereof at rs7733296 was more frequent in RA patients.

Individuals with PsA show a higher frequency of having one or more G alleles or the complementary allele(s) thereof (i.e., C allele(s)) compared to individuals with RA. Table 4 shows that individuals that are homozygous or heterozygous for the G allele (or the complementary allele; the C allele) at rs7733296 (i.e., the PDE4D gene) are more likely to have PsA and not RA.

TABLE 4
Association of SNP allele at rs7733296 and RA or PsA

| PDE4D (rs7733296) genotype | RA | PsA |
|---|---|---|
| C C | 1.2% | 2.1% |
| C T | 20.1% | 28.1% |
| T T | 78.7% | 69.8% |

The effect size (Odds Ratio, OR) of the two risk genotypes was calculated, using the non-risk genotype (TT or the complementary AA genotype) as baseline. Accordingly, the CC genotype (or the complementary GG genotype) had an OR (95% CI)=2.04 (1.02-4.06) (p-value 1-sided Fisher's test=0.03) and the CT genotype (or the complementary GA genotype) had an OR (95% CI)=1.57 (1.29-1.92) (p-value 1-sided Fisher's test=5.8e⁻⁶).

Further analysis of the genomic region of PDE4 identified another SNP associated with PsA vs. RA; rs9326069 (promoter of PDE4B locus) demonstrated a p-value of 0.009. The minor allele (i.e., the A allele) of rs9326069 was more frequent in PsA patients and the major allele (i.e., the G allele) was more frequent in RA patients. Neither the PDE4A nor PDE4C loci showed a significant association to the disease states.

For the PDE4B SNP at rs9326069, the effect sizes were not as strong as for PDE4D, yet the results show the A allele (or the complementary allele; the T allele) at rs9326069 was more frequent in individuals with PsA than individuals with RA. Here, the baseline non-risk genotype is GG (or the complementary CC genotype). For the data in Table 5, the AA genotype (or the complementary TT genotype) had an OR (95% CI)=1.27 (0.99-1.63) (p-value 1-sided Fisher's test=0.034) and AG genotype (or the complementary TC genotype) has an OR (95% CI)=1.13 (0.94-1.371 (n-value 1-sided Fisher's test=0.101.

TABLE 5
Association of SNP allele at rs9326069 and RA or PsA

| PDE4B (rs9326069) genotype | RA | PsA |
|---|---|---|
| A A | 16.3% | 18.7% |
| A G | 46.7% | 47.9% |
| G G | 37.0% | 33.4% |

The PsA-associated allelic variants described herein can be used to aid in the diagnosis of disease at the early stages when arthritis is less differentiated and more difficult to diagnose. The presence of one or two G alleles at rs7733296 and/or one or two A alleles at rs9326069 can be used to diagnose a patient as having PsA. Furthermore, a patient having PsA and one or more G alleles at rs7733296 and/or one or more A alleles at rs9326069 is likely to positively respond to a PDE4 inhibitor such as apremilast, cilomilast, diazepam, ibudilast, luteolin, mesembrenone, piclamilast, roflumilast, rolipram, or combinations thereof.

On the other hand, the presence of two A alleles at rs7733296 and/or two G alleles at rs9326069 can be used to diagnose a patient as having RA. Such a patient may be selected to receive an anti-TNFα inhibitor therapy. In some cases, it may be predicted that a patient previously diagnosed with RA and carrying the PsA-associated allelic variants will positively respond to a PDE4 inhibitor.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for aiding or assisting in the differential diagnosis of rheumatoid arthritis (RA) versus psoriatic arthritis (PsA) in an individual, the method comprising:
(a) detecting the presence of a single nucleotide polymorphism (SNP) in a cAMP-specific 3′,5′-cyclic phosphodiesterase 4 (PDE4) gene selected from the group consisting of a PDE4D gene, PDE4B gene, and combinations thereof in a sample obtained from the individual, wherein the SNP in the PDE4D gene comprises rs7733296 and the SNP in the PDE4B gene comprises rs9326069; and
(b) aiding or assisting in the diagnosis of (i) RA based on the presence of an A allele or a complementary allele thereof at rs7733296 and/or a G allele or a complementary thereof at rs9326069, or (ii) PsA based on the presence of a G allele or a complementary allele thereof at rs7733296 and/or an A allele or a complementary allele thereof at rs9326069.

2. The method of claim 1, wherein the diagnosis of RA is based on the presence of two A alleles or the complementary alleles thereof at rs7733296 and/or two G alleles or the complementary alleles thereof at rs9326069.

3. The method of claim 1, wherein the diagnosis of PsA is based on the presence of one or two G alleles or the complementary alleles thereof at rs7733296 and/or one or two A alleles or the complementary alleles thereof at rs9326069.

4. The method of claim 1, wherein the method improves the diagnosis of an early stage of RA or an early stage of PsA.

5. (canceled)

6. (canceled)

7. (canceled)

8. (canceled)

9. (canceled)

10. (canceled)

11. The method of claim 1, wherein step (a) comprises detecting the presence of a SNP allele in the PDE4D gene and a SNP allele in the PDE4B gene.

12. The method of claim 1, wherein the sample is selected from the group consisting of whole blood, plasma, serum, synovial fluid, saliva, tears, mucus, sweat, and urine.

13. A method for selecting an individual with rheumatoid arthritis (RA) or psoriatic arthritis (PsA) for treatment with a cAMP-specific 3′,5′-cyclic phosphodiesterase 4 (PDE4) inhibitor, the method comprising:
(a) detecting the presence of a single nucleotide polymorphism (SNP) in a PDE4 gene selected from the group consisting of a PDE4D gene, PDE4B gene, and combinations thereof in a sample obtained from the individual, wherein the SNP in the PDE4D gene comprises rs7733296 and the SNP in the PDE4B gene comprises rs9326069; and
(b) selecting the individual for treatment with the PDE4 inhibitor based on the presence of a G allele or a complementary allele thereof at rs7733296 and/or an A allele or a complementary allele thereof at rs9326069.

14. The method of claim 13, wherein the individual is selected for treatment with the PDE4 inhibitor based on the presence of two G alleles or the complementary alleles thereof at rs7733296 and/or two A alleles or the complementary alleles thereof at rs9326069.

15. The method of claim 13, wherein the PDE4 inhibitor is selected from the group consisting of apremilast, cilomilast, diazepam, ibudilast, luteolin, mesembrenone, piclamilast, roflumilast, rolipram, and combinations thereof.

16. (canceled)

17. (canceled)

18. (canceled)

19. (canceled)

20. (canceled)

21. (canceled)

22. (canceled)

23. The method of claim 13, wherein step (a) comprises detecting the presence of a SNP allele in the PDE4D gene and a SNP allele in the PDE4B gene.

24. The method of claim 13, wherein the sample is selected from the group consisting of whole blood, plasma, serum, synovial fluid, saliva, and urine.

25. A method for determining the likelihood that an individual with rheumatoid arthritis (RA) or psoriatic arthritis (PsA) will respond to treatment with a cAMP-specific 3′,5′-cyclic phosphodiesterase 4 (PDE4) inhibitor, the method comprising:
(a) detecting the presence of a single nucleotide polymorphism (SNP) in a PDE4 gene selected from the group consisting of a PDE4D gene, PDE4B gene, and combinations thereof in a sample obtained from the individual, wherein the SNP in the PDE4D gene comprises rs7733296 and the SNP in the PDE4B gene comprises rs9326069; and
(b) determining that the individual has a higher likelihood of response to treatment with the PDE4 inhibitor based on the presence of a G allele or a complementary allele thereof at rs7733296 and/or an A allele or a complementary allele thereof at rs9326069.

26. The method of claim 25, wherein the individual has a higher likelihood of response to treatment with the PDE4 inhibitor based on the presence of two G alleles or the complementary alleles thereof at rs7733296 and/or two A alleles or the complementary alleles thereof at rs9326069.

27. The method of claim 25, wherein the PDE4 inhibitor is selected from the group consisting of apremilast, cilomilast, diazepam, ibudilast, luteolin, mesembrenone, piclamilast, roflumilast, rolipram, and combinations thereof.

28. (canceled)

29. (canceled)

30. (canceled)

31. (canceled)

32. (canceled)

33. (canceled)

34. (canceled)

35. The method of claim 25, wherein step (a) comprises detecting the presence of a SNP allele in the PDE4D gene and a SNP allele in the PDE4B gene.

36. The method of claim 25, wherein the sample is selected from the group consisting of whole blood, plasma, serum, synovial fluid, saliva, and urine.

37. A method for treating rheumatoid arthritis (RA) comprising administering a therapeutically effective amount of a PDE4 inhibitor to a human subject suffering from RA and having a G allele or a complementary allele thereof at rs7733296 and/or an A allele or a complementary allele thereof at rs9326069.

38. The method of claim 37, wherein the human subject has two G alleles or the complementary alleles thereof at rs7733296 and/or two A alleles or the complementary alleles thereof at rs9326069.

39. (canceled)

40. (canceled)

41. (canceled)

42. A method for treating psoriatic arthritis (PsA) comprising administering a therapeutically effective amount of a PDE4 inhibitor to a human subject suffering from PsA and having a G allele or a complementary allele thereof at rs7733296 and/or an A allele or a complementary allele thereof at rs9326069.

43. The method of claim 42, wherein the human subject has two G alleles or the complementary alleles thereof at rs7733296 and/or two A alleles or the complementary alleles thereof at rs9326069.

44. (canceled)

45. (canceled)

46. (canceled)
